(19) United States
(12) Patent Application Publication
(10) Pub. No.: US 2021/0285024 A1
(43) Pub. Date: Sep. 16, 2021

(54) In vitro Screening Assay of TCPase Modulators

(71) Applicants: CENTRE NATIONAL DE LA RECHERCHE SCIENTIFIQUE (CNRS), Paris (FR); UNIVERSITE DE MONTPELLIER, Montpellier (FR)

(72) Inventors: Krzysztof ROGOWSKI, MONTPELLIER (FR); Siem VAN DER LAAN, CAZILHAC (FR)

(73) Assignees: CENTRE NATIONAL DE LA RECHERCHE SCIENTIFIQUE (CNRS), Paris (FR); UNIVERSITE DE MONTPELLIER, Montpellier (FR)

(21) Appl. No.: 17/259,970

(22) PCT Filed: Jul. 12, 2019
(86) PCT No.: PCT/EP2019/068871

§ 371 Date: Jan. 13, 2021

(30) Foreign Application Priority Data
Jul. 13, 2018 (EP) 18305958.3

(51) Int. Cl. C12Q 1/37

(52) U.S. Cl. CPC C12Q 1/37; G01N 2333/8103; G01N 2500/00

(57) ABSTRACT

The present invention concerns an in vitro screening assay for identification of modulators of tubulin carboxypeptidase (TCPase) activity comprising the steps of: (i) Contacting (a) a substrate of TCPase enzyme comprising an amino acids sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), and b) an isolated or recombinant TCPase enzyme; in the presence or absence (control) of the modulator compound to be tested, and under conditions for substrate cleavage, preferably detyrosination, and/or liberation of a C-terminal free aromatic amino acid residue, preferably a C-terminal free tyrosine; (ii) Using reagents for detecting and measuring the signal related to substrate cleavage, preferably detyrosination, and/or liberation of the C-terminal free aromatic amino acid residue, preferably the C-terminal free tyrosine; (iii) Measuring and comparing the level of substrate cleavage, preferably detyrosination and/or the level of the C-terminal free aromatic amino acid residue, preferably the C-terminal free tyrosine in presence and in absence (control) of the compound to be tested, and (iv) Selecting the modulators of TCPase for which the level of substrate cleavage, preferably detyrosination or liberation of the C-terminal aromatic amino acid residue, preferably C-terminal free tyrosine is increased in the presence of the compound to be tested (activators of TCPase) or decreased in the presence of the compound to be tested (inhibitors of TCPase). The present invention also concerns kits for performing such in vitro screening assay.

### FIELD OF THE INVENTION

The present invention relates to in vitro screening assay for identification of TCPase modulating compounds, in particular in vitro screening immuno-assay.

### BACKGROUND OF THE INVENTION

Microtubules (MTs) are the major types of cytoskeleton elements. They are formed by heterodimeric polymerization of two globular proteins called α- and β-tubulin. Within a cell, MTs grow, retract, and as such support numerous cellular processes including intracellular transport (cargo transport), cell motility, cell division, cell morphogenesis and mechanotransduction. Each particular MT function requires the recruitment of a specific set of MT associated proteins (MAPs) and molecular motors, many of which interact specifically with the C-terminal tails of tubulins that protrude from MT surface (Ciferri et al., 2008; Roll-Mecak and Vale 2008 (Nature)). One important way in which MTs are adapted to different functions is by alterations of the tubulin C-terminal tails through posttranslational modifications (PTMs). Almost half a century ago, detyrosination, i.e. the removal of the C-terminal tyrosine of alpha tubulins resulting in generation of so-called Δ1-tubulin, was the earliest reported tubulin PTMs. This PTM is reversible and addition of tyrosine is carried out by tubulin-tyrosine-ligase (TTL) which is already known for a long time. In contrast, tubulin detyrosinases have just very recently been identified (Aillaud et al 2017), allowing direct functional studies of tubulin detyrosination. The cycle of tyrosination/detyrosination is evolutionary conserved from unicellular early-branching eukaryotes such as Trypanosoma to humans (Preston et al., 1979).

In proliferating animal cells, the rapid MT turnover does not allow detyrosination to accumulate. As a result, the majority of MTs in interphasic cells are composed of tyrosinated tubulin while Δ1-tubulin is present only in a small subset of highly stable mostly perinuclear MTs. During mitosis, Δ1-tubulin is found exclusively in the peripheral regions of half spindles and it appears to be absent from astral fibers. Recent studies have shown that spindle detyrosination plays an important role in chromosome congregation during mitosis and is essential for meiotic drive. In addition, stable MT assemblages such as axonemes, centrioles and basal bodies, contain high levels of Δ1-tubulin. Furthermore, detyrosination has been found to play an essential role in generation of stable MT arrays during differentiation events such as cell polarization, myogenesis, and neurogenesis (Aillaud et al 2017). Thus, in general, detyrosination is associated with stable MTs.

Considering the key role of MTs for various cellular processes, it is not surprising that MT targeting compounds such as paclitaxel (taxol) are among most widely used drugs in anti-cancer therapy. The chemotherapeutic drug taxol is known to interact within a specific site on β-tubulin and as such stabilise microtubule and impact primarily dividing cells. However, MT-targeting drugs suffer from several drawbacks such as high toxicity and eventual development of drug-resistant cancer cells. For that reason, it is important to continue developing compounds that impact MT function. One novel approach, appears to be the targeting of the enzymes involved in tubulin posttranslational modifications, among which detyrosination seems hitherto to be the most promising venue.

Misregulation of tyrosination/detyrosination cycle of tubulin, frequently observed during cancer progression, is associated with increased tumor aggressiveness (Mialhe et al., 2001; Kato et al., 2004). The carboxyl-terminus of α-tubulin may be involved in the control of cell cycle progression (Maihato et al. 2015) and in the association/disassociation of motor proteins during cell division. The spindle-related defects are particularly interesting since several studies have shown that absence or downregulation of TTL activity is associated with cancer progression (Mialhe et al., 2001; Kato et al., 2004). Besides, accumulating studies show that microtubule-associated proteins can regulate taxol sensitivity in a wide range of cancer types.

Exploiting the alpha-tubulin sequence as a bait, two closely related proteins of the vasohibin family, VASH1 and VASH2 were identified as tubulin detyrosinases (Aillaud et al 2017; Nieuwenhuis et al., 2017). Interestingly, treatment of HEK293T cells deficient for VASH1 and VASH2 with Taxol, a microtubules stabilising agent, results in a robust increase in alpha-tubulin detyrosination demonstrating a yet unidentified activity (Nieuwenhuis et al., 2017). Fonrose et al. (2007) previously identified in cellulo putative inhibitors of tubulin carboxypeptidase activity, but the Applicant now demonstrated that these compounds were not capable of inhibit detyrosinase in the in vitro assay of the present invention likely reflecting the indirect action of these compounds on tubulin detyrosination using a cell-based assay. In particular, while SVBP-VASH2 complex strongly induced detyrosination in the assay, the in cellulo identified putative inhibitors of detyrosinase including parthenolide, alantolactone and costunolide in human HEK293 cells, did not result in any significant reduction of the detyrosinating activity. This suggests that the effect of these compounds in human cells on the level of detyrosination likely reflects an indirect adaptive mechanism that alters microtubule detyrosination more than direct binding and inhibition of the detyrosinating enzymes. So the in cellulo data disclosed by Fonrose et al. (2007), even combined with teaching of Aillaud et al. (2017) and Nieuwenhuis et al (2017) on VASH1 and VASH2 proteins would not help the skilled person to develop an improved in vitro assay, selective and specific due to the direct binding of the compounds and the enzymes, for identification of modulators of TCPase activity, object of the present invention.

The identification of specific modulators of enzymes with TCPase activity is of particular interest for treating disorders involving microtubule detyrosination, such as neurodegenerative diseases, neuronal regeneration disorders, cancers, muscular dystrophies, heart diseases, vascular disorders, retinal degeneration, infertility or ciliopathies. Importantly, the modulators can also be developed to target infectious parasites such as but not limited to Tryponosoma Brucei, Toxoplasma gondii and as such be designed to act on the parasite specific detyrosination enzyme and not on the human homolog.

Next to their use in pharmaceutical development, the modulators could also be used as tools to better understand the role of tubulin detyrosination in e.g. cancer occurrence, aggressiveness and progression. Overall, there is a strong need for compounds that may selectively modulate detyrosination mediated by a TCPase.

Detyrosinated tubulin was recently found to be decreased in fetal vessels of preeclampsia placentas. The reduction of placental detyrosination in preeclampsia could suggest a deficit in villous vascular plasticity and might be associated with the impaired arborization of the disease. In this setting, a compound that could activate tubulin carboxypeptidase activity, ie an activator of TCPase, could be of therapeutic interest.

Given the potential wide application of compounds targeting tubulin detyrosinases, there is still a need to develop a new method for in vitro screening of such compounds, preferably a high throughput screening (HTS) for detection of such compounds.

The present invention answers this expectation and proposes a method allowing detection of compounds that directly act on tubulin carboxypeptidases, in particular by high throughput screening (HTS). Validated hits can be identified within large chemical libraries and assay may serve lead optimization. The immuno-assay according to the invention is based on specifically generated antibodies raised to detect cleaved substrate of TCPase, in particular detyrosinated substrate of TCPase and identification of easy to produce substrate of TCPase containing a C-terminal tyrosine ‘Y’ including in particular polymerized microtubules, recombinant alpha-tubulin, engineered telokin protein (Q15746-8) and peptide use. The assay may be miniaturized and adapted to dedicated robotic workstations including microfluidic systems. The screen is reproducible, reliable, robust, and, most importantly, employ techniques that ensure there is reasonable confidence that the results will be biologically relevant.

The method of the present invention can be used for making a kit composed of all reagents necessary to perform the assay on standard plate reader equipment. The assay can easily be implemented in high throughput screening (HTS) methods for hit identification within large chemical libraries and following lead optimization in the pharmaceutical company. Moreover, the method of the present invention can also be used for making a kit composed of all reagents modulators of the activity on various different substrates (such as recombinant alpha-tubulin, purified tubulin dimers from diverse sources such as from Spodoptera frugiperda Sf9 cell culture, polymerized microtubule, telokin substrate and more).

### SUMMARY OF THE INVENTION

A first object of the present invention is an in vitro screening assay for identification of modulators of tubulin carboxypeptidase (TCPase) activity comprising the steps of:
(i) Contacting (a) a substrate of TCPase enzyme comprising an amino acids sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), and b) an isolated or recombinant TCPase enzyme; in the presence or absence (control) of the modulator compound to be tested, and under conditions for substrate cleavage, preferably detyrosination, and/or liberation of a C-terminal free aromatic amino acid residue, preferably a C-terminal free tyrosine;
(ii) Using reagents for detecting and measuring the signal related to substrate cleavage, preferably detyrosination, and/or liberation of the C-terminal free aromatic amino acid residue, preferably the C-terminal free tyrosine;
(iii) Measuring and comparing the level of substrate cleavage, preferably detyrosination and/or the level of the C-terminal free aromatic amino acid residue, preferably the C-terminal free tyrosine in presence, and in absence (control) of the compound to be tested, and
(iv) Selecting the modulators of TCPase for which the level of substrate cleavage, preferably detyrosination or liberation of the C-terminal free aromatic amino acid residue, preferably the C-terminal free tyrosine is increased in the presence of the compound to be tested (activators of TCPase) or decreased in the presence of the compound to be tested (inhibitors of TCPase).

In a particular embodiment, the in vitro screening assay is an immuno-assay, in particular a dot-blot or an ELISA assay, and more preferably a High Throughput Screening (HTS) assay based on ELISA assay.

Another object of the present invention is a kit for performing the in vitro screening immuno-assay, comprising reagents and instructions as disclosed hereunder.

Another object of the invention is a kit for performing the in vitro screening assay based on detection of a C-terminal aromatic residue via a colorimetric assay or using fluorescent properties of the aromatic residues, preferably a colorimetric assay using a tyrosinase to detect a C-terminal free tyrosine, comprising reagents and instructions as disclosed hereunder.

### DETAILED DESCRIPTION OF THE INVENTION

### Definitions

The ‘in vitro’ assay of the present invention distinguishes from an ‘in cellulo’ assay or ‘cell-based’ assay as disclosed in Fonrose et al.

As used herein, the term “contacting” intends bringing the reagents, ie TCPase enzyme and its substrate according to the invention, into close proximity with each other so that a chemical or biochemical reaction can occur among the reagents. In one aspect, the term intends admixing the components, either in a fluid reaction (ex: vessel) or on solid support (ex: plate).

The terms “modulators” of TCPase include any compound that interacts with or which modulates (increases or decreases) the activity of a TCPase or which modulates (increase or decrease) the binding of another compound to a TCPase. This includes agonists, antagonists, and their homologs and mimetics.

Inhibitors are compounds that, e.g., bind to, partially or totally block activity, decrease, prevent, delay activation, inactivate, desensitize, or downregulate TCPase or activity, e.g., antagonists.

Activators or enhancers are compounds that, e.g., bind to, stimulate, increase, open, activate, facilitate, enhance activation, sensitize, or upregulate TCPase or activity, e.g., agonists. TCPase modulators can include genetically modified versions of TCPase family members, e.g., with altered activity, as well as naturally occurring and synthetic ligands, antagonists, aptamers, agonists, small chemical molecules and the like.

In particular, the term ‘modulator of TCPase activity’ according to the present invention means a compound which directly alters the activity of the TCPase when brought into contact. During the course of the in vitro screening assay, the TCPase enzyme is put in contact with the substrate of TCPase in absence or presence of compounds to be tested, and the level of detyrosination is measured, for example by enzyme-linked immunosorbent assay (ELISA) system. In that context, a compound that reduces overall level of detyrosination compared to the control situation (in absence of the compound to be tested) is termed an ‘inhibitor’. Oppositely, a compound that increases overall level of detyrosination compared to the control situation is termed an ‘activator’.

The term “isolated” is used herein to refer to polypeptides and proteins that are separated from constituents, cellular and otherwise.

The peptides or proteins, described herein, may be isolated, or otherwise associated with structures or compounds to which they are not normally associated in nature, according to a variety of methods and processes known to those of skill in the art.

The term “purified” refers to a composition being substantially free from undesired contamination.

As used herein, “recombinant” refers to a polynucleotide synthesized or otherwise manipulated in vitro (e.g., “recombinant polynucleotide”), to methods of using recombinant gene products in cells or other biological systems to produce a polypetide; the recombinant polynucleotide or recombinant protein may be similar to natural polynucleotide or protein or not.

The term “engineered” refers to a polynucleotide or polypeptide obtained by in vitro human manipulations, such as for example insertion of nucleotide sequence into another nucleotide sequence to produce a recombinant engineered polypeptide comprising the both fused sequences.

The term “synthetic” refers to non-natural polynucleotide or polypeptide.

The term “polypeptide” refers to a compound of two or more amino acid subunits, amino acid analogs. The subunits may be linked by peptide bonds. As used herein the term “amino acid” refers to natural and/or unnatural or synthetic amino acids, amino acid analogs. A peptide of three or more amino acids is commonly called an oligopeptide if the peptide chain is short. If the peptide chain is long, the peptide is commonly called a polypeptide or a protein.

The amino acid sequences defined herein use the one letter code as following: A: Ala (alanine); R: Arg (arginine); N: Asn (asparagine); D: Asp (aspartic acid); C: Cys (cysteine); Q: Gln (glutamine); E: Glu (glutamic acid); G: Gly (glycine); H: His (histidine); I: Ile (isoleucine); L: Leu (leucine); K: Lys (lysine); M: Met (methionine); F:

Phe (phenylalanine); P: Pro (proline); S: Ser (serine); T: Thr (threonine); W: Trp (tryptophan); Y: Tyr (tyrosine); V: Val (valine).

The term “aromatic amino acid residue” refers to F: Phe (phenylalanine); W: Trp (tryptophan); Y: Tyr (tyrosine).

The proteins and peptides according to the invention also encompass “conservative variant” or “analog” which refer to a polypeptide which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. For example we may use the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W). In addition, individual substitutions, deletions or additions that alter, add or delete a single amino acid or a small percentage of amino acids in an encoded sequence can also be considered “conservatively modified variations.” Because of the degeneracy of the genetic code, a large number of functionally identical nucleic acids encode any given protein.

In particular, isolated or recombinant peptides from 5 to 50 amino acids (substrate of TCPase) according to the invention also encompass variants having at least 50%, 60%, 70%, 80%, 90%, 95%, 96%, 97%, 98%, 99% of sequence identity with the described amino acids sequences identified in the description as “SEQ ID No:”, with condition that the most C-terminal EEY or EDY or EAY or Y, preferably EEY or Y is 100% identity.

In particular, isolated or recombinant proteins (substrate of TCPase) according to the invention also encompass variants having at least 50%, 60%, 70%, 80%, 90%, 95% of sequence identity with the described amino acids sequences identified in the description as “SEQ ID No:”, with condition that the most C-terminal ends with an aromatic amino acid residue such as tyrosine (Y), phenylalanine (F) or tryptophan (W). In a preferred embodiment, the sequence ends with a C-terminal tyrosine residue.

The term ‘variant thereof’ in the rest of the description encompasses the above defined % of identity with the described amino acids sequences identified in the description as “SEQ ID No:”.

The percent identities referred to in the context of the disclosure of the present invention are determined on the basis of a global alignment of sequences to be compared, i.e., on an alignment of the sequences taken in their entirety over their entire length using any algorithm well-known to a person skilled in the art, such as the algorithm of Needleman and Wunsch (1970). This sequence comparison may be performed using any software well-known to a person skilled in the art, for example the Needle software by using the “Gap open” parameter equal to 10.0, the “Gap extend” parameter equal to 0.5 and a “Blosum 62” matrix. The Needle software is for example available on the website ebi.ac.uk under the name “Align”.

The term “substrate cleavage” means the proteolysis of the substrate by the TCPase enzyme, in particular the cleavage of the ultimate C-terminal aromatic amino acid residue, preferably the tyrosine (Y) residue (also named “detyrosination”).

In Vitro Screening Assay

A first object of the invention is an in vitro screening assay for identification of modulators of tubulin carboxypeptidase (TCPase) activity comprising the steps of:
(i) Contacting (a) a substrate of TCPase enzyme comprising an amino acids sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), and b) an isolated or recombinant TCPase enzyme; in the presence or absence (control) of the modulator compound to be tested, and under conditions for substrate cleavage, preferably detyrosination, and/or liberation of a C-terminal free aromatic amino acid residue, preferably a C-terminal free tyrosine;
(ii) Using reagents for detecting and measuring the signal related to substrate cleavage, preferably detyrosination, and/or liberation of a C-terminal free aromatic amino acid residue, preferably a C-terminal free tyrosine;
(iii) Measuring and comparing the level of substrate cleavage, preferably detyrosination and/or the level of the C-terminal free aromatic amino acid residue, preferably the C-terminal free tyrosine in presence and in absence (control) of the compound to be tested, and
(iv) Selecting the modulators of TCPase for which the level of substrate cleavage, preferably detyrosination or liberation of the C-terminal free aromatic amino acid residue, preferably the C-terminal free tyrosine is increased in the presence of the compound to be tested (activators of TCPase) or decreased in the presence of the compound to be tested (inhibitors of TCPase).

The steps (i) to (iv) of the screening generally follow a sequential order as disclosed above but some of the steps may occur simultaneously or with another order than the one illustrated above.

Substrates of TCPase Enzymes

In a particular embodiment of the invention, the substrate of TCPase enzyme comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), is selected from the group consisting of:
a) Purified or recombinant α-tubulin,
b) Recombinant engineered telokin,
c) Isolated or recombinant peptide from 4 to 50 amino acid residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y),
d) Isolated or recombinant microtubules,
e) Isolated or recombinant tubulin dimers of alpha and beta tubulin,
f) Microtubule-associated proteins (MAP) such as microtubule-associated proteins RP/EB family members 1 to 3,
g) or
h) Mixtures thereof.

Non-limitative examples of such TCPase enzyme substrates as defined above and comprising a most C-terminal aromatic amino acid residue, preferably a tyrosine ‘Y’ and used according to the present invention are disclosed in the following Table 1:

TABLE 1

| SEQ | | | | |
|---|---|---|---|---|
| ID | Sequence | | Uniprot | |
| NO: | description | Origin | Reference | Function |
| 17 | Fully tyrosinated | Artificial | Artificial | Engineered Telokin (Uniprot |
| | variant | | | Q6PDN3 -4). The C-terminal |
| | Telokin/Mus | | | sequence of alpha-tubulin |
| | musculus | | | (Tubulin alpha-1A chain - human) |
| | | | | has been introduced in the |
| | | | | Telokin sequence. Fully |
| | | | | tyrosinated variant. |
| 19 | Tubulin alpha-1A | Homo sapiens | Q71U36 | Tubulin alpha-1A chain. Tubulin is |
| | chain | | | the major constituent of |
| | | | | microtubules. It binds two moles |
| | | | | of GTP, one at an exchangeable |
| | | | | site on the beta chain and one at |
| | | | | a non-exchangeable site on the |
| | | | | alpha chain. |
| 20 | Tubulin alpha-1B | Homo sapiens | P68363 | Tubulin alpha-1B chain. |
| | chain | | | |
| 21 | Tubulin alpha-1C | Homo sapiens | Q9BQE3 | Tubulin alpha-1C chain. |
| | chain | | | |
| 22 | Tubulin alpha- | Homo sapiens | Q13748 | Tubulin alpha-3C/D chain. |
| | 3C/D chain | | | |
| 23 | Tubulin alpha-3E | Homo sapiens | Q6PEY2 | Tubulin alpha-3E chain |
| | chain | | | |
| 24 | Tubulin alpha-8 | Homo sapiens | Q9NY65 | Tubulin alpha-8 chain |
| | chain | | | |
| 25 | Tubulin alpha | Trypanosoma | Q4GYY5 | Tubulin alpha chain |
| | chain | brucei | | |
| 26 | Tubulin beta | Trypanosoma | Q4GYY6 | Tubulin beta chain |
| | chain | brucei | | |
| 27 | Tubulin alpha | Toxoplasma | P10873 | Tubulin alpha chain |
| | chain | gondii | | |
| 28 | Tubulin alpha | Spodoptera | G3CKA7 | Tubulin alpha chain. Spodoptera |
| | chain | frugiperda | | frugiperda. |
| 29 | Tubulin alpha | Drosophila | P06605 | Tubulin alpha chain |
| | chain | melanogaster | | |
| 30 | Microtubule- | Homo sapiens | Q15691 | Microtubule-associated protein |
| | associated | | | RP/EB family member 1. May be |
| | protein RP/EB | | | involved in spindle function by |
| | family member 1 | | | stabilizing microtubules and |
| | | | | anchoring them at centrosomes |
| 31 | Microtubule- | Homo sapiens | Q15555 | Microtubule-associated protein |
| | associated | | | RP/EB family member 2. May be |
| | protein RP/EB | | | involved in microtubule |
| | family member 2 | | | polymerization, and spindle |
| | | | | function by stabilizing |
| | | | | microtubules and anchoring them |
| | | | | at centrosomes. May play a role |
| | | | | in cell migration |
| 32 | Microtubule- | Homo sapiens | Q9UPY8 | Microtubule-associated protein |
| | associated | | | RP/EB family member 3 : acts as |
| | protein RP/EB | | | a regulator of minus-end |
| | family member 3 | | | microtubule organization: |
| | | | | interacts with the complex formed |
| | | | | by AKAP9 and PDE4DIP, leading |
| | | | | to recruit CAMSAP2 to the Golgi |
| | | | | apparatus, thereby tethering non- |
| | | | | centrosomal minus-end |
| | | | | microtubules to the Golgi, an |
| | | | | important step for polarized cell |
| | | | | movement |
| 33 | 50 amino acids | Artificial | 1A/1B | KRAFVHWYVGEGMEEGEFSEA |
| | Last C-ter alpha- | | | REDMAAL |
| | tubulin 1A/1B | | | EKDYEEVGVDSVEGEGEEEGE |
| | | | | EY |
| 34 | 12 amino acids | Artificial | 1A/1B | VEGEGEEEGEEY |
| | Last C-ter alpha- | | | |
| | tubulin 1A/1B | | | |
| 35 | 12 amino acids | Artificial | 3C | SVEAEAEEGEEY |
| | Last C-ter alpha- | | | |
| | tubulin 3C | | | |
| 36 | 12 amino acids | Artificial | 1C | DSADGEDEGEEY |
| | Last C-ter alpha- | | | |
| | tubulin 1C | | | |
| 37 | 12 amino acids | Artificial | 3E | SVEAEAEEGEAY |
| | Last C-ter alpha- | | | |
| | tubulin 3E | | | |
| 38 | 8 amino acids | Artificial | 1A/1B | GEEEGEEY |
| | last C-ter alpha- | | | |
| | tubulin 1A/1B | | | |
| 39 | 11 amino acids | Artificial | 1A/1B | EGEGEEEGEEY |
| | last C-ter alpha- | | | |
| | tubulin 1A/1B | | | |

Recombinant Engineered Telokin as Preferred Substrate (Containing C-Terminal Tubulin Sequence)

In a particular and preferred embodiment, the substrate of TCPase is a recombinant engineered telokin having at least the last 4 amino acids residues of the C-terminal sequence of an α-tubulin and a tyrosine (Y) as ultimate C-terminal amino acid residue.

In particular, a recombinant engineered telokin comprises the most C-terminal tail of alpha-tubulin (the 11 most C-terminal residues EGEGEEEGEEY=SEQ ID NO: 39).

Telokin protein (Q15746, SEQ ID NO: 15, Homo sapiens) is a small isoform of myosin-light chain kinase protein family. The protein function is involved in calcium/calmodulin-dependent myosin light chain kinase implicated in smooth muscle contraction via phosphorylation of myosin light chains (MLC). The specific Telokin isoform (Q6PDN3-4, SEQ ID NO: 16, Mus musculus) is transcribed from an alternative promoter resulting in the usage of Met-1761 as initiator codon. It has no catalytic activity and is highly produced in bacteria. The use of telokin gene to modify it's ending in any potential substrate (human alpha tubulins, trypanosoma alpha and beta tubulin etc.) make it a substrate of choice to adapt by molecular engineering. A recombinant engineered tyrosinated telokin has been obtained as illustrated further in the description (Examples).

In a particular and preferred embodiment, the substrate of TCPase is a recombinant tyrosinated telokin comprising the amino acid sequence SEQ ID NO:17 (Mus musculus, fully tyrosinated variant) or a variant thereof. In another embodiment, the substrate of TCPase is a recombinant telokin comprising Trypanosoma Brucei alpha tubulin (SEQ ID NO: 25) or Trypanosoma Brucei beta tubulin (SEQ ID NO: 26).

Additional Substrates of TCPase Enzyme

As an alternative, the substrate of TCPase according to the invention is selected from isolated or recombinant microtubules (MTs), which are formed by the polymerization of a dimer of two globular proteins, α- and β-tubulin heterodimers.

Tubulin dimers can be extracted and purified from any eukaryotic cell system such as human cell lines (HEK, Hela, RPE, MCF-7, etc.), mouse cell-line (C2C12, etc.), Trypanosoma Brucei, Spodoptera frugiperda cell line (Sf9, Sf21, etc.), and many more. Obtained tubulin dimers should be stored at −80° C. and used for generating microtubules (MTs) with compounds that lower the critical concentration (glycerol, GTP etc.). Tubulin polymerizations were generally carried out in 80 mM PIPES pH 6.9, 0.5 mM EGTA, 2 mM MgCl2, 1 mM GTP, and the reactions occur at 37° C. (FIG. 7).

By ‘recombinant microtubules’ or ‘synthetic microtubules’ according to the invention, it means microtubules that are chemically constructed, for example by methods of synthetic biology, including solid phase peptide synthesis (SPPS), prior thiol capture strategy or native chemical ligation (NCL).

As an alternative, the substrate of TCPase according to the invention is selected from isolated or recombinant binding protein, in particular an isolated or recombinant microtubule-associated protein (“MAP proteins”) RP/EB from human. In particular, mention may be made of microtubule-associated protein RP/EB family member 1 comprising the amino acid sequence SEQ ID NO:30, microtubule-associated protein RP/EB family member 2 comprising the amino acid sequence SEQ ID NO: 31 or microtubule-associated protein RP/EB family member 3 comprising the amino acid sequence SEQ ID NO: 32, or variants thereof.

As an alternative, the substrate of TCPase according to the invention is selected from isolated or recombinant tubulin dimers, in particular purified tubulin dimers obtained from any eukaryotic cell containing tubulin, such as human cell lines (HEK, Hela, RPE, MCF-7, etc.), mouse cell-line (C2C12, etc.), Trypanosoma Brucei, Spodoptera frugiperda cell line (Sf9, Sf21, etc.).

Purified or Recombinant α-Tubulin as Other Preferred Substrates

In a first and preferred embodiment, the tubulin dimers are purified tyrosinated tubulin from Spodoptera Frugiperda (Sf) cells (Sf9, Sf21 etc.). While searching for a reliable source of tyrosinated tubulin we found that tubulin purified from Spodoptera Frugiperda (Sf) cells (Sf9, Sf21 etc.) represented an unlimited source of fully tyrosinated tubulin (FIG. 1). Tubulin was purified using a TOG column (Widlund et al. 2012 MBoC) and analyzed by mass spectrometry. Analysis of untreated Sf9 purified tubulin by mass spectrometry did not detect any detyrosinated alpha-tubulin. This purified tubulin of Spodoptera Frugiperda is polymerization competent and can be used as substrate in in vitro detyrosination assays. So the tubulin purified from Sf9 cells represents an unlimited source of polymerization competent fully tyrosinated tubulin.

In a particular embodiment of the invention, the substrate of TCPase is a purified or recombinant tyrosinated tubulin comprising the amino acid sequence of SEQ ID NO:28 (Uniprot G3CKA7, Spodoptera Frugiperda) or variant thereof. The purified or recombinant tyrosinated tubulin of Spodoptera Frugiperda is advantageous for use in the in vitro assay of the present invention, in comparison to tubulin purified from HEK cells, as it does not bare significant amount of tubulin modification (FIG. 18).

In another particular and preferred embodiment, the α-tubulin is a recombinant alpha tubulin (α-tubulin) which can be produced in bacteria according to known methods. In particular, we may use a purified or recombinant α-tubulin from human in particular comprising the amino acid sequences SEQ ID NO: 19 to SEQ ID NO: 24), or from Trypanosoma brucei in particular comprising the amino acids sequences SEQ ID NO: 25 and SEQ ID NO: 26, or from Toxoplasma gondii in particular comprising the amino acids sequence SEQ ID NO: 27, or from Drosophila melanogaster in particular comprising the amino acids sequence (SEQ ID NO: 29).

Isolated or Recombinant Peptides as Other Preferred Substrates

As another alternative, the substrate of TCPase according to the invention is selected from isolated or recombinant peptides from 4 to 50 amino acid residues, in particular 5 to 20 amino acids, preferably 6 to 12 amino acid residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), in particular comprising an amino acid sequence selected from SEQ ID NO: 33 to SEQ ID NO: 39 or variant thereof.

In a particular embodiment, the substrate of TCPase according to the invention is a peptide GEEEGEEY (SEQ ID NO: 38).

The substrate of TCPase described above may be use alone or in mixture.

In a particular embodiment, the substrate of TCPase enzyme is selected from:
purified or recombinant tubulin from Spodoptera Frugiperda (Sf) cells, in particular from Sf9 cells, in particular comprising the amino acid sequence of SEQ ID NO:28 (Uniprot G3CKA7, Spodoptera Frugiperda) or variant thereof;
recombinant telokin comprising a C-terminal sequence of alpha-tubulin, in particular recombinant tyrosinated telokin comprising the amino acid sequence SEQ ID NO: 17 or variant thereof, and
recombinant peptides from 5 to 20 amino acids residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), in particular recombinant peptide comprising the amino acid sequence SEQ ID NO: 33 to SEQ ID NO: 39 or variants thereof.

In a particular and preferred embodiment, the substrate of TCPase enzyme is selected from purified or recombinant tubulin from Sf9 cells, in particular comprising the amino acid sequence of SEQ ID NO:28 (Uniprot G3CKA7, Spodoptera Frugiperda) or variant thereof.

In a particular and preferred embodiment, the substrate of TCPase enzyme is selected from recombinant engineered telokin, in particular comprising an amino acid sequence SEQ ID NO: 17 or variant thereof.

In a particular embodiment, the substrate of TCPase according to the invention is a peptide GEEEGEEY (SEQ ID NO: 38).

TCPase Enzymes or TCPase Proteins

In the context of the present invention, the terms ‘TCPase enzyme’ or ‘TCPase protein’ or ‘protein having a tubulin carboxypeptidase activity’ or ‘protein having a TCP activity’ are used for referring to a class of proteins that are able to cleave off the ultimate C-terminal aromatic amino acid residue, in particular the tyrosine to release the C-terminal aromatic amino acid residue, in particular C-terminal tyrosine residue (Y) from a native or recombinant substrate, in particular a native or recombinant tyrosinated tubulin or preferably a recombinant engineered tyrosinated telokin.

In the rest of the description, we will use the terms ‘substrate cleavage’ or in particular ‘detyrosination’, and the terms ‘cleaved substrate’ or in particular ‘detyrosinated substrate’.

Non-limitative examples of such TCPase enzyme used according to the present invention are disclosed in the following Table 2:

TABLE 2

| SEQ ID | Sequence | | Uniprot | |
|---|---|---|---|---|
| NO: | description | Origin | Reference | Function |
| 1 | Ubiquitin carboxyl- | Homo | P54578 | Indispensable for synaptic |
| | terminal hydrolase 14 | sapiens | | development and function at |
| | | | | neuromuscular junctions (NMJs). |
| 2 | Ubiquitin carboxyl- | Homo | P45974 | Thiol-dependent hydrolysis of ester, |
| | terminal hydrolase 5 | sapiens | | thioester, amide, peptide and |
| | | | | isopeptide bonds formed by the C- |
| | | | | terminal Gly of ubiquitin (a 76- |
| | | | | residue protein attached to proteins |
| | | | | as an intracellular targeting signal). |
| 3 | Methionine | Homo | P50579 | The enzyme is active with cobalt, |
| | aminopeptidase 2 | sapiens | | zinc, manganese or divalent iron |
| | | | | ions. Most likely, methionine |
| | | | | aminopeptidases function as |
| | | | | mononuclear Fe2+- |
| | | | | metalloproteases under |
| | | | | physiological conditions, and the |
| | | | | catalytically relevant metal-binding |
| | | | | site has been assigned to the |
| | | | | histidine-containing high-affinity site. |
| 4 | Xaa-Pro | Homo | Q9NQW7 | Catalyzes the removal of a |
| | aminopeptidase 1 | sapiens | | penultimate prolyl residue from the |
| | | | | N-termini of peptides, such as Arg- |
| | | | | Pro-Pro. |
| 5 | Tripeptidyl-peptidase 2 | Homo | P29144 | May be able to complement the 26S |
| | | sapiens | | proteasome function to some extent |
| | | | | under conditions in which the latter |
| | | | | is inhibited. |
| 6 | Vasohibin-1 | Homo | Q7L8A9 | Angiogenesis inhibitor. Inhibits |
| | | sapiens | | migration, proliferation and network |
| | | | | formation by endothelial cells as well |
| | | | | as angiogenesis. |
| 7 | Dihydropyrimidinase- | Homo | Q14194 | Necessary for signaling by class 3 |
| | related protein 1 | sapiens | | semaphorin and subsequent |
| | (Collapsin response | | | remodeling of the cytoskeleton. |
| | mediator protein 1- | | | Plays a role in axon guidance, |
| | CRMP1) | | | invasive growth and cell migration. |
| | | | | May participate in cytokinesis. |
| 8 | Dihydropyrimidinase- | Homo | Q16555 | Plays a role in neuronal |
| | related protein 2 | sapiens | | development and polarity, as well as |
| | | | | in axon growth and guidance, |
| | | | | neuronal growth cone collapse and |
| | | | | cell migration. Necessary for |
| | | | | signaling by class 3 semaphorins |
| | | | | and subsequent remodeling of the |
| | | | | cytoskeleton. May play a role in |
| | | | | endocytosis |
| 9 | Dihydropyrimidinase- | Homo | Q14195 | Necessary for signaling by class 3 |
| | related protein 3 | sapiens | | semaphorins and subsequent |
| | | | | remodeling of the cytoskeleton. |
| | | | | Plays a role in axon guidance, |
| | | | | neuronal growth cone collapse and |
| | | | | cell migration |
| 10 | Dihydropyrimidinase- | Homo | 014531 | Necessary for signaling by class 3 |
| | related protein 4 | sapiens | | semaphorins and subsequent |
| | | | | remodeling of the cytoskeleton. |
| | | | | Plays a role in axon guidance, |
| | | | | neuronal growth cone collapse and |
| | | | | cell migration |
| 11 | Dihydropyrimidinase- | Homo | Q9BPU6 | May have a function in neuronal |
| | related protein 5 | sapiens | | differentiation and/or axon growth. |
| 12 | Vasohibin-2 | Homo | Q86V25 | Angiogenesis inhibitor. Inhibits |
| | | sapiens | | network formation by endothelial |
| | | | | cells. |
| 13 | Small vasohibin- | Homo | Q8N300 | Enhances the solubility and |
| | binding protein | sapiens | | secretion of VASH1 and prevents its |
| | | | | ubiquitination. Also enhances |
| | | | | secretion of VASH2 |
| 14 | Uncharacterized | Trypanosoma | Q387E4 | |
| | protein/VASH | brucei | | |

In particular, the TCPase enzyme according to the invention is selected from proteins having at least 30%, 40%, 50%, 60%, 70%, 80%, 90%, 95%, 99%, amino acid sequence identity with the amino acid sequence selected from SEQ ID NO: 1 (Ubiquitin carboxyl-terminal hydrolase 14), SEQ ID NO: 2 (Ubiquitin carboxyl-terminal hydrolase 5), SEQ ID NO: 3 (Methionine aminopeptidase 2), SEQ ID NO: 4 (Xaa-Pro aminopeptidase 1), SEQ ID NO: 5 (Tripeptidyl-peptidase 2), SEQ ID NO: 6 (Vasohibin-1), SEQ ID NO: 7 (Dihydropyrimidinase-related protein 1), SEQ ID NO: 8 (Dihydropyrimidinase-related protein 2), SEQ ID NO:9 (Dihydropyrimidinase-related protein 3), SEQ ID NO: 10 (Dihydropyrimidinase-related protein 4), SEQ ID NO: 11 (Dihydropyrimidinase-related protein 5), SEQ ID NO: 12 (Vasohibin-2), or SEQ ID NO: 14 (Uncharacterized protein/VASH of Trypanosoma brucei), preferably SEQ ID NO: 6 (Vasohibin-1) or SEQ ID NO: 12 (Vasohibin-2) in presence or absence of Small vasohibin-binding protein (SEQ ID NO: 13).

In a particular embodiment, the isolated proteins with a tubulin carboxypeptidase activity are obtained from a brain extract, such as a brain extract from pigs, and the mass spectrometric data are aligned with human reference sequences, in order to identify corresponding human proteins.

In a particular embodiment, a) the substrate of TCPase enzyme is selected from purified or recombinant α-tubulin from parasites, in particular comprising an amino acid sequence selected from SEQ ID NO: 25 and SEQ ID NO: 26 (Trypanosoma brucei) or variants thereof, and b) the isolated or recombinant TCPase enzyme is selected from parasites proteins, in particular having at least 30%, 40%, 50%, 60%, 70%, 80%, 90%, 95%, 99%, amino acid sequence identity with the amino acid sequence SEQ ID NO: 14 (Uncharacterized protein/VASH of Trypanosoma brucei).

The term ‘parasite’ encompasses living organism on (ectoparasite) or in (endoparasite) another organism that feeds at the expense of the host. Mainly, any eukaryotic pathogen that is deemed emerging or re-emerging such as Plasmodium or Neospora and the trypanosomatides. As examples of parasites that may induce human infections, mention may be made of Trypanosoma, Toxoplasma, Leishmania, Ameobe, Giardia lamblia, Trichomonas, Microsporidia . . . .

In a particular and preferred embodiment, a) the substrate of TCPase enzyme is a recombinant engineered telokin, in particular comprising an amino acid sequence SEQ ID NO: 17 or variant thereof, and b) the isolated or recombinant TCPase enzyme is selected from proteins having at least 30%, 40%, 50%, 60%, 70%, 80%, 90%, 95%, 99%, amino acid sequence identity with the amino acid sequence SEQ ID NO: 6 (Vasohibin-1) or SEQ ID NO: 12 (Vasohibin-2) in presence or absence of Small vasohibin-binding protein SVBP (SEQ ID NO: 13). In presence of SVBP, we call about SVBP-VASH complex.

Each of the above listed TCPase (table 2) may be combined with each of the above listed substrate of TCPase (table 1) in the in vitro assay of the present invention.

In a particular an preferred embodiment, mention may be made of the following combinations in the in vitro assay of the present invention.

TABLE 3

| Substrates | | Recombinant | Isolated or |
|---|---|---|---|
| & | Purified tubulin | engineered | recombinant |
| TCPases | from Sf cells | Telokin | peptides |
| VASH-1 | x | x | x |
| VASH-1 + | x | x | x |
| SVBP | | | |
| VASH-2 | x | x | x |
| VASh-2 + | x | x | x |
| SVBP | | | |

The said substrates and TCPases listed in table 3 are defined above.

Conditions for Substrate Cleavage, in Particular Detyrosination

The man skilled in the art will adapt and optimize the concentrations of each reagents, in particular substrate of TCPase and TCPase enzyme to favor the substrate cleavage reaction, in particular the detyrosination reaction.

In a particular embodiment, the concentration of TCPase enzymes or fractions thereof contacted with substrate of TCPase is in the range of 0.1 μM to 1 mM, preferably 0.25 μM to 500 μM, more preferably 0.5 μM to 300 μM, and even more preferably 0.5 μM to 200 μM, in order to provide optimal conditions for substrate cleavage (e.g detyrosination) of the substrate of TCPase.

In a particular embodiment, the reaction temperature is maintained in the range of 1° C. to 70° C., preferably 5° C. to 65° C., more preferably 10° C. to 60° C., even more preferably 15° C. to 55° C., most preferably 19° C. to 43° C., and for example 19° C. to 37° C. in order to provide optimal conditions for the putative TCPase enzyme to cleave the C-terminal aromatic amino acid residue, in particular to detyrosinate the substrate of TCPase.

The man skilled in the art will use the in vitro methods of screening available and adapted to detection of modulators of TCPase by measuring substrate cleavage, in particular detyrosination.

Detection of Cleaved C-Terminal Free Aromatic Residue

In a first embodiment, the step (ii) of the in vitro screening assay of the invention comprises detection of the C-terminal free aromatic amino-acid via a colorimetric assay or using fluorescent properties of the aromatic residues, preferably detection of the C-terminal free-tyrosine via a colorimetric assay using a tyrosinase.

In a preferred embodiment, the in vitro screening assay is a tyrosinase-based assay, in particular a colorimetric tyrosinase-based assay. In such an assay according to the invention, step (ii) comprises the use of a tyrosinase and conditions for detecting and measuring free-tyrosine.

Such assay is developed by applying well-described tyrosinase assays that allow quantitative measurements of free tyrosine in a sample (Park et al. 2003, journal of protein chemistry). Tyrosinase is a multicatalytic enzyme participating in several enzymatic reactions in melanin synthetic pathway: the hydroxylation of tyrosine to 3,4-dihydroxyphenylalanine (DOPA), the oxidation of DOPA to DOPAquinone, and the oxidation of 5,6-dihydroxyindole to 5,6-dihydroxyquinone.

In a particular embodiment, we measured dynamic changes of dopaquinone content in the reaction by spectrophotometric analysis. Tyrosinase catalyzes metabolism of both L-tyrosine and L-dopa to dopaquinone.

The original aspect of such assay is the use of a TCPase enzyme, in particular VASH2 and a tyrosine containing substrate such as microtubules, recombinant alpha-tubulin protein, recombinant tyrosinated Telokin or peptides according to the present invention.

The use of recombinant tyrosinase that enzymatically oxidize tyrosine to a colorimetric intermediate. Tyrosinase is a polyphenol oxidase that is the rate-limiting enzyme for controlling the production of melanin, which is then possible to measure with a standard 96/384/1536 spectrophotometric plate reader. Samples are compared to a known concentration of tyrosine standard within the microtiter plate format.

In a particular embodiment, the substrate of TCPase is a recombinant engineered telokin (SEQ ID NO: 17), the enzyme TCPase is vasohibin-2 (VASH2) (SEQ ID NO: 12) in the presence or absence of SVBP (SEQ ID NO: 13), and the step (ii) of detecting and measuring the level of free-tyrosine comprises a colorimetric assay, in particular an assay detecting dopaquinone as a result of tyrosinase activity. As such the levels of dopaquinone depend on the TCPase mediated detyrosination leading to an increase in the level of free-tyrosine and as such in the conversion to dopaquinone due to the excess presence of tyrosinase.

In another particular embodiment, the in vitro screening assay is based on a non-enzymatic detection method, for detecting cleaved C-terminal free tryptophan (W). Detection of tryptophan can be achieved by fluorescent analysis specific for the aromatic side chain, such as the one disclosed in Gordon et al. (1949).

Immuno-Assay (Detection of Detyrosinated Substrate)

In another and preferred embodiment, the in vitro screening assay according to the invention is an immunoassay.

As used herein, the term ‘immunoassay’ is an assay based on the antigen-antibody binding specificity which allows detecting and optionally quantifying antigens (such as cleaved substrate or detyrosinated substrate) or antibodies. Immunoassays can be classified as immunoassays using non-labeled reagents (antigens or antibodies) and immunoassays using labeled reagents wherein the reagent (antigen or antibody) is labeled with a marker, such as a radioactive isotope, an enzyme, a fluorophore or a substance involved in a (chemo) luminescent reaction, giving rise to so-called radioimmunoassays (e.g., RIA, IRMA), enzyme immunoassays (e.g., EMIT, CEDIA, ELISA for enzyme-linked immunosorbent assay), fluoroimmunoassays (e.g., FPIA, SLFIA, FETI, DELFIA) or luminescent immunoassays, respectively.

In a particular embodiment of the invention, said immunoassay is an ELISA.

In another particular embodiment of the invention, said immunoassay is a Dot-blot.

The phrase “specifically (or selectively) binds” to an antibody, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. In the context of the present invention, the ‘antigen’ is ‘cleaved substrate’ in particular ‘detyrosinated substrate’ which is recognized by an antibody raised against said ‘cleaved substrate’ in particular ‘detyrosinated substrate’.

Included among the immunoassays that use labeled reagents are homogeneous immunoassays and heterogeneous immunoassays; the latter comprise performing one or more steps of separating the antigen or antibody not bound to the other member of the binding pair (antibody or antigen) which will generally be bound to a solid phase or support, for example, microplates, magnetic particles, nitrocellulose membranes, etc. Furthermore, depending on the design of the assay, heterogeneous immunoassays can be competitive or non-competitive, depending on if the labeled reagent (antigen or antibody) is added in a limited amount or in excess, respectively.

Immunoassays allow identifying antigens (direct techniques) or antibodies against an antigen (indirect techniques).

In the context of the present invention, the ‘antigen’ or ‘target protein or peptide’ is a cleaved substrate of TCPase, in particular detyrosinated substrate of TCPase, ie a substrate of TCPase cleaved or detyrosinated by the TCPase enzyme present in the reaction solution, in presence or absence (control) of the compounds to be tested (putative modulators of TCPase), and under conditions known by persons skilled in the art allowing the formation of a cleaved substrate of TCPase-antibody complex, preferably detyrosinated substrate of TCPase-antibody complex. This antibody is named ‘detection antibody’, distinct from ‘capture antibody’ that may be fixed on solid support.

The detection antibody may be labelled or coupled to a marker.

As it is used herein, the term “marker” relates to an indicator reagent which allows detecting the cleaved or detyrosinated substrate of TCPase-detection antibody complex. By way of non-limiting illustration, said marker can be an enzyme (e.g., peroxidase, glycosidase, alkaline phosphatase, glucose-6-phosphate dehydrogenase, β-galactosidase, β-glucosidase, β-glucuronidase, etc.), a fluorescent compound or fluorophore (e.g., fluorescein, rhodamine, etc.), a (chemo)luminescent compound (e.g., dioxetanes, acridiniums, phenanthridiniums, ruthenium, luminol, etc.), radioactive elements (sulfur, iodine, etc.), etc.

In a particular embodiment, said marker is a peroxidase.

The selection of a particular marker is not critical provided it is able to produce a signal by itself or together with one or more additional substances.

By way of illustration, when the marker is an enzyme the detection of the complex can be carried out by contacting said complex with a suitable substrate, and optionally with suitable enzymatic amplification agents and/or activators. Illustrative examples of said substrates include for example for peroxidases:

Chromogenic: substrates based on 2,2-azinobis(3-ethylbenzthiazoline-6-sulfonic) (ABTS) acid, o-phenylenediamine (OPD), 3,3′,5,5′-tetramethylbenzidine (TMB), o-dianisidine, 5-aminosalicylic acid, 3-dimethylaminobenzoic (DMAB) acid and 3-methyl-2-benzothiazolinehydrazone (MBTH), 3-amino-9-ethylcarbazole (AEC) and 3,3′-diaminobenzidine (DAB) tetrachloride, etc.

Fluorogenic: 4-hydroxy-3-methoxyphenylacetic acid, reduced phenoxazines and reduced benzothiazines, including Amplex® Red reagent, Amplex UltraRed reagent, reduced dihydroxanthens, etc.

In a particular embodiment, said marker is a peroxidase, such as the horseradish peroxidase, and the chromogenic substrate is TMB.

Indirect Technique (e.g Indirect ELISA)

In a first embodiment of the invention, the substrate of TCPase (which will give the ‘target cleaved or detyrosinated substrate’ in presence of TCPase enzyme) is directly or indirectly immobilized on a support, such as a well of a microtiter plate, magnetic beads, non-magnetic beads, columns, matrices, membranes, etc.

These materials can be used in suitable forms, such as films, sheets, plates, etc., or it can be used to coat inert carriers (e.g., paper, glass, plastic films, etc.). The immobilization of the substrate of TCPase (which will give the ‘target cleaved or detyrosinated substrate’ in presence of TCPase enzyme) on said support includes ionic, hydrophobic, and/or similar interactions.

The said substrate of TCPase will be recognized by a detection antibody specific to cleaved or detyrosinated substrate of TCPase. This detection antibody may be labelled or coupled to a secondary labelled antibody specific to first antibody.

Direct Technique (e.g Direct ELISA)

In another embodiment of the invention, the capture antibody specific to cleaved or detyrosinated substrate of TCPase is directly or indirectly immobilized on a support, such as a well of a microtiter plate, magnetic beads, non-magnetic beads, columns, matrices, membranes, as disclosed above. The said substrate of TCPase will be recognized by a detection antibody specific to cleaved or detyrosinated substrate of TCPase. This detection antibody may be labelled or coupled to a secondary labelled antibody specific to first antibody

The detection antibodies specific to said ‘cleaved target or detyrosinated substrate’ are incubated with said ‘cleaved target or detyrosinated substrate’ for a time period and under conditions suitable in order for cleaved substrate-antibody complexe or detyrosinated substrate-antibody complexes to be formed.

Furthermore, if desired, positive and negative controls can be used for putting the immunoassay of the invention into practice.

In a particular embodiment, Epoxy-Y compound is used as positive control.

In a particular embodiment, non-tyrosinated isolated or recombinant telokin such as the one comprising the amino acid sequence SEQ ID NO: 15, SEQ ID NO: 16 or SEQ ID NO: 18, is used as a negative control.

In a particular embodiment of the invention, the immunoassay is an enzyme immunoassay, a fluoroimmunoassay, a luminescent immunoassay or a radioimmunoassay, preferably an enzyme immunoassay (ELISA).

Enzyme-Linked Immunosorbent Assay ELISA (Preferred One):

In a particular and preferred embodiment, the in vitro method of screening of modulators of TCPase according to the invention is an ELISA method. This immunoassay utilizes an antibody labeled with an enzyme marker such as horseradish peroxidase.

In a usual immunoassay ELISA, samples, including a standard containing protein of interest, control specimens, and unknowns, are pipetted into these wells. During the first incubation, the protein antigen binds to the solid support or a capture antibody. After washing, a detection antibody is added to the wells, and this antibody binds to the immobilized protein captured during the first incubation. After removal of excess detection antibody, an HRP conjugate (secondary antibody or streptavidin) is added and binds to the detection antibody. After a third incubation and washing to remove the excess HRP conjugate, a substrate solution is added and is converted by the enzyme to a detectable form (color signal). The intensity of this colored product is directly proportional to the concentration of antigen present in the original specimen. ELISA substrates differ in ease-of-use, sensitivity (i.e., lower limit of detection) and compatibility with imaging equipment. Colorimetric, chemiluminescent and fluorescent ELISA substrates are usable in the context of the test.

In a particular embodiment of the invention, standardized amount of substrate of TCPase according to the invention, preferably engineered Telokin protein containing the C-terminal sequence of alpha-tubulin as disclosed above is coated to a multiwell plate (96/384/1536). Recombinant TCPase enzyme, in particular VASH2 is brought into contact with the substrate in absence or presence of the compound to be tested for a specific time. The enzyme is removed and the well is abundantly and repeatedly washed with a mild detergent solution to remove all VASH2 proteins. HRP-coupled antibody directed against the detyrosinated Telokin engineered substrate is incubated in each well and excess appropriately washed off with a mild detergent solution to remove antibodies that are non-specifically bound. After the final wash step, the plate is developed by adding Chromogenic HRP substrate (the exact type of substrate used for detection will depend on the particular assay and mode of detection) is added and microplate measurement is performed accordingly. High sensitivity may be reached by using classical enhancers (many different enhancers exist and may increase signal profoundly).

In a particular embodiment, the step (ii) of the in vitro screening immuno-assay of the invention comprises:
adding an effective amount of specific labelled antibody raised against cleaved substrate, in particular detyrosinated alpha-tubulin (dTyr-Ab), under conditions that favor the formation of a complex antibody—cleaved substrate; and
means for revealing the labelled signal;
or alternatively
adding an effective amount of a primary specific antibody raised against cleaved substrate, in particular detyrosinated alpha-tubulin (dTyr-Ab), under conditions that favor the formation of a complex antibody—cleaved substrate;
adding an effective amount of a secondary labelled antibody specific of the primary antibody, under conditions that favor the formation of a complex primary antibody-cleaved substrate-labelled secondary antibody, and
means for revealing the labelled signal,
and wherein the reaction occurs in soluble (fluid) phase or solid phase.

In a particular embodiment of the invention, the antibody is labelled with a marker selected in the group consisting of an enzyme, a fluorescent compound or fluorophore, a (chemo)luminescent compound or a radioactive element, preferably an enzyme and more preferably a peroxidase.

Soluble or Fluid Phase

The in vitro assay and reaction may occur in soluble phase (fluid vessel) or solid phase. By ‘solid phase’ or ‘solid support’ according to the invention, it encompasses for example, microplates, magnetic particles, nitrocellulose membranes, etc.

In a particular embodiment, microplates are used as solid support.

In a particular embodiment of the invention, the immuno-assay occurs in solid phase, and wherein:
substrate of TCPase is coated on solid support, in particular on microplate, and the TCPase enzyme, the compound(s) to be tested and the antibodies raised against cleaved substrate, in particular detyrosinated α-tubulin (dTyr-Ab), optionally labelled or combined with labelled secondary antibodies raised against primary antibodies are added in the reaction solution, or
alternatively, the antibodies raised against cleaved substrate, in particular detyrosinated α-tubulin (dTyr-Ab), are coated on solid support, in particular on microplate, and TCPase enzyme, substrate of TCPase, the compound(s) to be tested, and secondary labelled antibodies raised against cleaved substrate, are added in the reaction solution, or
alternatively, the TCPase enzyme is coated on solid support, in particular on microplate, and the substrate of TCPase, the compound(s) to be tested, and the antibodies raised against cleaved substrate, in particular detyrosinated α-tubulin (dTyr-Ab), optionally labelled or combined with labelled secondary antibodies raised against primary antibodies, are added in the reaction solution.

In a particular and preferred embodiment of in vitro immuno-assay of the invention, (a) the substrate of TCPase comprises dimers of alpha-tubulin extracted from SF9 cells (Spodoptera frugiperda origin), in particular comprising the amino acid sequence SEQ ID NO:27 or a variant thereof, (b) the TCPase enzyme is vasohibin-2 (VASH2) (SEQ ID NO: 12) in the presence or absence of SVBP (SEQ ID: 13), and the step (ii) of detecting and measuring the level of detyrosinated-substrate comprises at least an antibody raised against detyrosinated α-tubulin SF9 (dTyr-Ab SF9) coupled to horseradish peroxidase (HRP).

Dot Blot/Slot Blot

In another embodiment, the immunoassay according to the invention is a dotblot. In this technique, a reaction mixture containing the substrate and the enzyme in absence or in presence of the compound is directly spotted onto the filter. The filter is allowed to dry and the membrane is incubated in blocking buffer to prevent non-specific binding. Similarly to ELISA, HRP-coupled antibody directed against the cleaved substrate is incubated over the membrane, excess is washed-off and the membrane is developed using e.g. chromogenic HRP substrate. Intensities of the signals on autoradiograms were quantified by densitometric scanning. Alternatively, we designed a method using a slot blot approach. The steps are identical as for the dot blot approach, this is the samples are applied to a nitrocellulose or PVDF membrane with a vacuum manifold to produce an orderly grid of samples. Once dry, dot blots and slot blots are subjected to the same immunodetection steps used for Western blotting.

Microfluidic

In another embodiment, the immunoassay according to the invention is a microfluidic assay.

The ELISA assay can be adapted to microfluidic equipment/platform. Those miniaturized analytical platforms are capable of manually performing parallel liquid transfer, reaction, and analysis. Multiple examples exist of miniaturized ELISA tests.

High Throughput Screening (HTS)

In a particular embodiment, the in vitro screening assay is a High Throughput Screening (HTS) assay, in particular a High Throughput Screening (HTS) assay based on ELISA assay. HTS is a method for scientific experimentation especially used in drug discovery and relevant to the fields of biology and chemistry (High-throughput screening assays for the identification of chemical probes. Inglese J, et al. Nat Chem Biol. 2007 August; 3(8):466-79. Review). Using robotics, data processing/control software, liquid handling devices, and sensitive detectors, high-throughput screening allows a researcher to quickly conduct millions of chemical, genetic, or pharmacological tests. Through this process one can rapidly identify active compounds, antibodies, or genes that modulate a particular biomolecular pathway. The results of these experiments provide starting points for drug design and for understanding the interaction or role of a particular biochemical process in biology.

In a particular embodiment, the invention provides solid phase based in vitro assays in a high throughput format, where the TCPase or substrates of TCPase (ie microtubules, alpha-tubulin, tubulin dimers, recombinant tyrosinated telokin, peptides . . . ) are attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 1000 to about 1500 different compounds. It is also possible to assay multiple compounds in each plate well. It is possible to assay several different plates per day; assay screens for up to about 6,000-20,000 different compounds are possible using the integrated systems of the invention. Also, microfluidic reagent manipulation may be used.

The spectrum of low-throughput screening (10,000-50,000 assay points) medium-throughput screening (50,000-100,000 data points) and high-throughput screening (100,000-500,000 data points) can be defined. The scale of implementation of a given screen is greatly influenced by format, application of technology (e.g. automation), time and resource constraints.

Antibodies

As used herein, the terms “antibody,” “antibodies” includes whole antibodies and any antigen binding fragment or a single chain thereof. Thus the term “antibody” includes any protein or peptide containing molecule that comprises at least a portion of an immunoglobulin molecule. The terms “antibody,” “antibodies” also include immunoglobulins of any isotype, fragments of antibodies which retain specific binding to antigen, including, but not limited to, Fab, Fab′, F(ab)2, Fv, scFv, dsFv, Fd fragments, dAb, VH, VL, VhH, and V-NAR domains; minibodies, nanobodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues.

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, rabbit, etc. As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous supply of a specific monoclonal antibody. Alternative techniques for generating or selecting antibodies include in vitro exposure of lymphocytes to antigens of interest, and screening of antibody display libraries in cells, phage, or similar systems.

In the context of the present invention, specific antibodies directed against cleaved or detyrosinated tubulin were generated. The antibody was raised by injecting rabbits with a peptide composed of the very C-terminal sequence of detyrosinated tubulin (without the most C-terminal Y (DeTyr)). In a particular and preferred embodiment, we use a Spodoptera frugiperda (SF) tubulin, wherein the sequence of the peptide contains the 7 most C-terminal sequence of the protein without the most C-terminal Y (DeTyr) and an additional cysteine at the N-terminal for practical reasons (Seq: C-EGEGAEE).

Characterization of the antibody is performed by expression of full-length mouse or Spodoptera frugiperda alpha-tubulin or detyrosinated counterpart in HEK cells (FIG. 6).

Kit

The present invention also concerns the kits comprising the reagents and instructions for performing the in vitro screening assay disclosed above.

In particular, a kit of the invention comprises substrates TCPases (preferably lyophilized), reaction buffer, substrate solvent, stabilized enzyme TCPases in appropriate long-storage buffer, specific antibodies directed against cleaved or detyrosinated substrate coupled to detection system (e.g. HRP) (for immuno-assay), a compound known to inhibit TCPase (positive control), a standard (negative control) and a detailed protocol.

The kit may be used as a solution-based assay featuring the speed, high sensitivity, and convenience required for screening TCPase modulators in a high-throughput format (96/384/1536).

The TCPases and substrates of TCPase used in the kit are preferably the combinations listed in the Table 3 disclosed above.

In particular, the substrate of TCPase is preferably selected from:
(i1) a purified or recombinant tubulin from Spodoptera Frugiperda (Sf) cells, in particular from Sf9 cells, in particular comprising the amino acid sequence of SEQ ID NO:28 (Uniprot G3CKA7, Spodoptera Frugiperda) or variant thereof;
(i2) a recombinant telokin comprising a C-terminal sequence of alpha-tubulin, in particular recombinant tyrosinated telokin comprising the amino acid sequence SEQ ID NO: 17 or variant thereof, and
(i3) recombinant peptides t from 5 to 20 amino acids residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), in particular recombinant peptide comprising the amino acid sequence SEQ ID NO: 33 to SEQ ID NO: 39 or variants thereof.

And the TCPase enzyme according to the invention is preferably selected from proteins having at least 30%, 40%, 50%, 60%, 70%, 80%, 90%, 95%, 99%, amino acid sequence identity with the amino acid sequence selected from SEQ ID NO: 6 (Vasohibin-1) or SEQ ID NO: 12 (Vasohibin-2) in presence or absence of Small vasohibin-binding protein (SEQ ID NO: 13).

In a particular embodiment, the invention concerns a kit for performing the in vitro screening immuno-assay of the invention, comprising:
(i) a TCPase enzyme substrate as defined in the present invention, preferably a recombinant engineered telokin as substrate of TCPase enzyme, in particular comprising an amino acid sequence SEQ ID NO: 17 or variant thereof,
(ii) a TCPase enzyme as defined in the present invention, preferably an isolated or recombinant TCPase enzyme selected from proteins having at least 30%, 40%, 50%, 60%, 70%, 80%, 90%, 95%, 99%, amino acid sequence identity with the amino acid sequence of Vasohibin-2 (SEQ ID NO: 12) in presence or absence of Small vasohibin-binding protein (SEQ ID NO: 13),
(iii) antibodies raised against cleaved substrate, preferably detyrosinated α-tubulin (dTyr-Ab), in particular raised against detyrosinated α-tubulin SF9 (dTyr-Ab SF9) and coupled to horseradish peroxidase (HRP), and optionally secondary antibodies raised against the cleaved substrate antibodies, preferably the detyrosinated α-tubulin antibodies,
(iv) a negative control, in particular comprising a non-tyrosinated isolated or recombinant telokin,
(v) a positive control, in particular comprising Epoxy-Y compound,
(vi) a fluid vessel or alternatively a solid support for coating or pre-coating either the substrate of TCPase either the TCPase enzyme, preferably membranes or microplates,
(vii) reagents for allowing contact of said substrate with TCPase enzyme in reaction conditions for substrate cleavage, preferably detyrosination,
(viii) reagents for detecting and measuring the level of substrate cleavage, preferably detyrosination; and
(ix) optionally a notice of use.

Another object of the invention is a kit for performing the in vitro screening assay for detection of the free C-terminal aromatic amino-acid via a colorimetric assay or using fluorescent properties of the aromatic residues, comprising:
(i) a TCPase enzyme substrate, preferably a recombinant engineered telokin as substrate of TCPase enzyme, in particular comprising an amino acid sequence SEQ ID NO: 17 or variant thereof,
(ii) a TCPase enzyme, preferably an isolated or recombinant TCPase enzyme selected from proteins having at least 30%, 40%, 50%, 60%, 70%, 80%, 90%, 95%, 99%, amino acid sequence identity with the amino acid sequence of Vasohibin-2 (SEQ ID NO: 12) in presence or absence of Small vasohibin-binding protein (SEQ ID NO: 13),
(iii) optionally a tyrosinase for the detection of a C-terminal free-tyrosine via colorimetric assay,
(iv) a fluid vessel or alternatively a solid support for coating or pre-coating either the substrate of TCPase either the TCPase enzyme, preferably membranes or microplates,
(v) reagents for allowing contact of said substrate with TCPase enzyme in reaction conditions for liberation of a C-terminal free-aromatic amino acid residue, preferably a C-terminal free-tyrosine,
(vi) reagents for detecting and measuring the level of the C-terminal free-aromatic amino acid residue, preferably the C-terminal free-tyrosine; and (vii) optionally a notice of use.

In another particular embodiment, the invention concerns a kit for performing the in vitro screening immuno-assay of the invention, comprising:
(i) a purified or recombinant tubulin from Spodoptera frugiperda Sf cells as substrate of TCPase enzyme, in particular comprising an amino acid sequence SEQ ID NO: 28 or variant thereof,
(ii) an isolated or recombinant TCPase enzyme selected from proteins having at least 30%, 40%, 50%, 60%, 70%, 80%, 90%, 95%, 99%, amino acid sequence identity with the amino acid sequence of Vasohibin-2 (SEQ ID NO: 12) in presence or absence of Small vasohibin-binding protein (SEQ ID NO: 13),
(iii) antibodies raised against cleaved substrate, preferably detyrosinated α-tubulin (dTyr-Ab), in particular raised against detyrosinated α-tubulin SF9 (dTyr-Ab SF9) and coupled to horseradish peroxidase (HRP), and optionally secondary antibodies raised against the cleaved substrate antibodies, preferably the detyrosinated α-tubulin antibodies,
(iv) a negative control, in particular comprising a non-tyrosinated isolated or recombinant telokin,
(v) a positive control, in particular comprising Epoxy-Y compound,
(vi) a fluid vessel or alternatively a solid support for coating or pre-coating either the substrate of TCPase either the TCPase enzyme, preferably membranes or microplates,
(vii) reagents for allowing contact of said substrate with TCPase enzyme in reaction conditions for substrate cleavage, preferably detyrosination,
(viii) reagents for detecting and measuring the level of substrate cleavage, preferably detyrosination; and
(ix) optionally a notice of use.

Another object of the invention is a kit for performing the in vitro screening assay for detection of the free C-terminal aromatic amino-acid via a colorimetric assay or using fluorescent properties of the aromatic residues, comprising:
(i) a purified or recombinant tubulin from Spodoptera frugiperda Sf cells as substrate of TCPase enzyme, in particular comprising an amino acid sequence SEQ ID NO: 28 or variant thereof,
(ii) an isolated or recombinant TCPase enzyme selected from proteins having at least 30%, 40%, 50%, 60%, 70%, 80%, 90%, 95%, 99%, amino acid sequence identity with the amino acid sequence of Vasohibin-2 (SEQ ID NO: 12) in presence or absence of Small vasohibin-binding protein (SEQ ID NO: 13),
(iii) optionally a tyrosinase for the detection of a C-terminal free-tyrosine via colorimetric assay,
(iv) a fluid vessel or alternatively a solid support for coating or pre-coating either the substrate of TCPase either the TCPase enzyme, preferably membranes or microplates,
(v) reagents for allowing contact of said substrate with TCPase enzyme in reaction conditions for liberation of a C-terminal free-aromatic amino acid residue, preferably a C-terminal free-tyrosine,
(vi) reagents for detecting and measuring the level of the C-terminal free-aromatic amino acid residue, preferably the C-terminal free-tyrosine; and (vii) optionally a notice of use.

In another particular embodiment, the invention concerns a kit for performing the in vitro screening immuno-assay of the invention, comprising:
(i) recombinant peptides from 5 to 20 amino acids residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), in particular recombinant peptide comprising the amino acid sequence SEQ ID NO: 33 to SEQ ID NO: 39 or variants thereof,
(ii) an isolated or recombinant TCPase enzyme selected from proteins having at least 30%, 40%, 50%, 60%, 70%, 80%, 90%, 95%, 99%, amino acid sequence identity with the amino acid sequence of Vasohibin-2 (SEQ ID NO: 12) in presence or absence of Small vasohibin-binding protein (SEQ ID NO: 13),
(iii) antibodies raised against cleaved substrate, preferably detyrosinated α-tubulin (dTyr-Ab), in particular raised against detyrosinated α-tubulin SF9 (dTyr-Ab SF9) and coupled to horseradish peroxidase (HRP), and optionally secondary antibodies raised against the cleaved substrate antibodies, preferably the detyrosinated α-tubulin antibodies,
(iv) a negative control, in particular comprising a non-tyrosinated isolated or recombinant telokin,
(v) a positive control, in particular comprising Epoxy-Y compound,
(vi) a fluid vessel or alternatively a solid support for coating or pre-coating either the substrate of TCPase either the TCPase enzyme, preferably membranes or microplates,
(vii) reagents for allowing contact of said substrate with TCPase enzyme in reaction conditions for substrate cleavage, preferably detyrosination,
(viii) reagents for detecting and measuring the level of substrate cleavage, preferably detyrosination; and
(ix) optionally a notice of use.

Another object of the invention is a kit for performing the in vitro screening assay for detection of the free C-terminal aromatic amino-acid via a colorimetric assay or using fluorescent properties of the aromatic residues, comprising:
(i) recombinant peptides from 5 to 20 amino acids residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), in particular recombinant peptide comprising the amino acid sequence SEQ ID NO: 33 to SEQ ID NO: 39 or variants thereof,
(ii) an isolated or recombinant TCPase enzyme selected from proteins having at least 30%, 40%, 50%, 60%, 70%, 80%, 90%, 95%, 99%, amino acid sequence identity with the amino acid sequence of Vasohibin-2 (SEQ ID NO: 12) in presence or absence of Small vasohibin-binding protein (SEQ ID NO: 13),
(iii) optionally a tyrosinase for the detection of a C-terminal free-tyrosine via colorimetric assay,
(iv) a fluid vessel or alternatively a solid support for coating or pre-coating either the substrate of TCPase either the TCPase enzyme, preferably membranes or microplates,
(v) reagents for allowing contact of said substrate with TCPase enzyme in reaction conditions for liberation of a C-terminal free-aromatic amino acid residue, preferably a C-terminal free-tyrosine,
(vi) reagents for detecting and measuring the level of the C-terminal free-aromatic amino acid residue, preferably the C-terminal free-tyrosine; and (vii) optionally a notice of use.

The in vitro screening assay can easily be implemented in high throughput screening (HTS) methods for hit identification within large chemical libraries and following lead optimization in the pharmaceutical company.

Therapeutic Applications

Detyrosination of microtubules is associated with cancer progression, aberrant neuronal networks, weak neuronal remodeling, plasticity and/or adaptation.

Accordingly, the use of such modulators of TCPase, selected by the in vitro screening assay of the present invention, may have a positive impact in the treatment of disorders involving microtubule detyrosination, in particular heart disorder, vascular disorder, cancers, neurodegenerative disorders, muscle disorders, infertility, ciliopathies, more generally a disorder involving altered microtubule detyrosination in an animal, preferably but not limited to a mammal. For instance, inhibitors of TCPase may be used for increasing the microtubule dynamics and thereby impacting neuroregeneration, constituting good candidates for treating neurodegenerative diseases, preferably selected from Alzheimer disease, Parkinson disease, psychiatric disorders, and neural disorders, neuronal regeneration disorders, cancers, preferably selected from colon cancer and neuroblastoma, muscle disorders such as muscular dystrophies, retinal degeneration, heart diseases, vascular disorders, infertility, and ciliopathies (Cell 2018 review Magiera et al.). In another and particular application, the use of such modulators of TCPase from parasites may help finding a treatment against human parasites. The term ‘parasite’ encompasses living organism on (ectoparasite) or in (endoparasite) another organism that feeds at the expense of the host. Mainly, any eukaryotic pathogen that is deemed emerging or re-emerging such as Plasmodium or Neospora and the trypanosomatids. As examples of parasites that may induce human infections, mention may be made of Trypanosoma, Toxoplasma, Ameobe, Giardia lamblia, Trichomonas, Microsporidia . . . In particular, such modulators compounds selected from the in vitro screening assay according to the present invention may be use in a pharmaceutical composition for the prevention and/or treatment of disorders related to alterations of TCPase activity, in particular neurodegenerative disorders or muscular disorders, in particular Alzheimer disease, Parkinson disease and/or dementia, cancer, Duchenne myopathy dystrophy, Chronic obstructive pulmonary disease (COPD).

### DESCRIPTION OF THE FIGURES

FIG. 1: Schematic representation of tubulin purification procedure from Spodoptera Frugiperda (SF) cells (Insect cells). Tubulin was mock- or carboxypeptidase treated and analyzed on by gel electrophoresis. Next tubulin was in-gel digested using trypsin (0.5 μg/band; Gold; Promega). Peptides were then analyzed online by nano-flow HPLC-nanoelectrospray ionization using a Qexactive Plus mass spectrometer (Thermo Fisher Scientific) coupled to a nano-LC system (U3000-RSLC, Thermo Fisher Scientific).

FIG. 2: Coomassie staining of samples collected at various steps during the purification protocol of bacterially produced CRMP1 recombinant protein. Purification consists of immobilized metal affinity chromatography (IMAC). Arrow indicates recombinant CRMP1 protein.

FIG. 3: Coomassie staining of samples collected at various steps during the two-step purification protocol of bacterially produced VASH1 recombinant protein. A first step consisting of immobilized metal affinity chromatography (IMAC) was followed by ion exchange (IEX) chromatography in order to further increase purity of recombinant protein. Arrow indicates recombinant VASH1 protein.

FIG. 4: Coomassie staining of samples collected at various steps during the two-step purification protocol of bacterially produced VASH2 recombinant protein. A first step consisting of immobilized metal affinity chromatography (IMAC) was followed by ion exchange (IEX) chromatography in order to further increase purity of recombinant protein. Arrow indicates recombinant VASH2 protein.

FIG. 5: Coomassie staining of samples collected at various steps during the two-step purification protocol of bacterially produced Trypanosoma brucei VASH (TbVASH) recombinant protein. A first step consisting of immobilized metal affinity chromatography (IMAC) was followed by ion exchange (IEX) chromatography in order to further increase purity of recombinant protein. Arrow indicates recombinant TbVASH protein.

FIG. 6: Characterization of the Spodoptera frugiperda (Sf) antibody. The antibody was generated by injection of the detyrosinated tubulin sequence in rabbits (Seq: C-EGEGAEE). Immunoblot analysis of HEK cells transfected with indicated tubulin constructs. Twenty-four hours post transfection samples were collected and analyzed by immunoblot with indicated antibodies. Immunoblot analysis showed that the mouse (Mm) antibody failed to recognize Spodoptera Frugiperda (Sf) detyrosinated tubulin and inversely

FIG. 7: Polymerization of tubulin into microtubules. Microscopy analysis of rhodamine labeled microtubules. Polymerization was achieved in presence of 20 μM Taxol at 37° C. during 30 minutes. Representative imaged of the obtained microtubules fibers.

FIG. 8: VASH2 acts as autonomous TCPase. In vitro detyrosination assay using recombinant VASH1 and VASH2 proteins alone or coexpressed with Small Vasohibin

Binding Protein (SVBP). In vitro detyrosination was performed using Spodoptera Frugiperda (Sf) purified tubulin.

FIG. 9: Activation of TCPase by SVBP. In cellulo (HEK) detyrosination assay using coexpression of VASH1 and VASH2 without or in presence of Small Vasohibin Binding Protein (SVBP). Coexpression of SVBP induces VASH1 and VASH2 detyrosination activity.

FIG. 10: Recombinant human VASH2 produced in bacteria detyrosinates polymerized microtubules (MT), tubulin dimers (TD) and recombinant alpha tubulin. Oppositely, VASH1 was much less effective on microtubules and tubulin dimers and almost inactive on recombinant alpha tubulin produced in bacteria.

FIG. 11: Recombinant human VASH2 and catalytically dead (VASH2D) produced in bacteria detyrosinates a 8 amino acid peptide consisting of GEEEGEEY. The samples were doted on nitrocellulose and assessed for detyrosination of the peptide using a specific antibody detecting detyrosination.

FIG. 12: Time course analysis of VASH2-mediated detyrosination. In vitro detyrosination assay of bacterially purified VASH2. Reactions were stopped at indicated time and analyzed by immunoblot and relative optical density was measured.

FIG. 13: Immuno-assay based detyrosination assay. In vitro detyrosination assay using recombinant VASH2 brought in contact with increasing dose (0.1; 1 and 10 μM) of previously described peptide-based inhibitor (Epoxy-Y). The assay is performed using Spodoptera Frugiperda (Sf) purified tubulin.

FIG. 14: Schematic representation of preferred HTS immuno-assay. The substrate coated 96 wells plate is first brought into contact, in absence (control) or presence of a compound to be tested, with the recombinant active bacterially produced VASH2 for 15 minutes. The enzymes is washed off using appropriate buffer (preferred is phosphate buffer), and HRP-coupled antibody specifically targeting detyrosinated tubulin is brought in contact with substrate. Excess antibody is washed off and detection is performed in plate reader.

FIG. 15: Colorimetric analysis of tyrosine concentration in the reaction mixture. (A) shows the ideal excitation wavelength (300 nm). (B) Linearity of colorimetric signal obtained when free tyrosine is measured at 305 nm. The concentration of tyrosine is displayed in molarity.

FIG. 16: Colorimetric analysis of free tyrosine released by VASH2 detyrosination of recombinant human alpha tubulin. OD305 was measured of control samples (filled histogram) and VASH2-mediated tyrosine release.
A. control sample containing tyrosinase,
B. control sample containing recombinant human alpha tubulin,
C. control samples containing tyrosinase (Tyr) and VASH2 as detyrosinase,
D reaction mixture containing recombinant human alpha tubulin, VASH2 mediating tyrosine release and tyrosinase to allow detection of free tyrosine.
OD305 was measured 30 min after onset of reaction at room temperature.

FIG. 17: Detyrosination assay performed using tubulin purified from Sf9 cells and recombinant SVBP-VASH2 complex in absence or presence of putative inhibitors of detyrosinase. FIG. 17a: putative inhibitors of detyrosinase disclosed in Fonrose et al (2007). FIG. 17b: Western blot. FIG. 17c: Representation of relative optical density (detyrosination/tubulin).

FIG. 18: Detyrosination assay performed using human tubulin purified from HEK293 cells and recombinant SVBP-VASH2 complex. FIG. 18a: Western blot. FIG. 18b: Representation of relative optical density (detyrosination/tubulin).

FIG. 19: Detyrosination assay performed using Sf tubulin and recombinant Trypanosoma brucei VASH protein (Western blot).

The present invention will be illustrated by the following non-limitative examples.

### EXAMPLES

Material and Methods

Preparation of Substrates of TCPase:

Preferred Substrate of TCPase:

In the context of the present invention, a recombinant tyrosinated telokin has been obtained as following: the C-terminal tail of alpha-tubulin (the 11 most C-terminal residues EGEGEEEGEEY=SEQ ID NO: 39) has been engineered at the C-terminal part of the Telokin isoform (Q6PDN3-4Q15746-8). Mouse cDNA was obtained using prime script RT-PCR kit (Takara) and telokin was amplified using adapted forward and reverse primers containing cloning sites. The amplicon was extracted and purified and the expression plasmid as well as the amplicon digested adapted restriction enzymes for cloning in bacterial expression vectors (pET-28a(+) and pGEX). In particular the both sequences are recombinantly expressed in a host cell, such as an E. coli. Obtained plasmid containing the last C-terminal amino acid sequence of human alpha tubulin 1A (N-EGEGEEEGEEY=SEQ ID NO: 39) at the very end of the telokin gene was sequence verified by double digestion diagnostics and by sequencing. Next, bacteria were transformed and selected on an antibiotic containing LB-dish. A single antibiotic-resistant colony was picked and grown to confluency as a starter overnight. Next day, fresh LB medium was inoculated with the bacteria and optical density (OD600) measured at regular interval. Once the bacteria OD600 reached between 0.4 and 0.6, IPTG was added for inducing protein production. After 4 hours, cells were harvested and telokin substrate purified using IMAC technology.

We obtained a recombinant tyrosinated telokin comprising the sequence SEQ ID NO:17 (Mus musculus, fully tyrosinated variant).

Negative control Telokin lacking the most C-terminal tyrosine (Y) residue can easily be implemented. In particular, we used an isolated (non-tyrosinated) telokin comprising the sequence SEQ ID NO:15 (Homo sapiens, Uniprot Q15746-8) or SEQ ID NO:16 (Mus musculus, Uniprot Q6PDN3-4) or a recombinant (detyrosinated V1 variant) telokin comprising the amino acid sequence SEQ ID NO: 18 (the C-terminal sequence of alpha-tubulin (Tubulin alpha-1A chain—human) without the ultimate Y, has been introduced in the Telokin sequence). Various substrates can be engineered to generate additional needed controls in the assay.

Additional Substrates of TCPase:
Purified or recombinant tyrosinated tubulin from Spodoptera Frugiperda (Sf) cells (Sf9, Sf21 etc.): Tubulin was purified using a TOG column (Widlund et al. 2012 MBoC) and analyzed by mass spectrometry. In particular, we used a purified or recombinant tyrosinated tubulin comprising the amino acid sequence of SEQ ID NO:28 (Uniprot G3CKA7); Tubulin was polymerized to obtain fully purified microtubules.
Recombinant alpha tubulin (α-tubulin) produced in bacteria according to known methods: in particular, we used a purified or recombinant α-tubulin from human in particular comprising the amino acid sequences SEQ ID NO: 19 to SEQ ID NO: 24), or from Trypanosoma brucei in particular comprising the amino acids sequences SEQ ID NO: 25 and SEQ ID NO: 26, or from Toxoplasma gondii in particular comprising the amino acids sequence SEQ ID NO: 27, or from Drosophila melanogaster in particular comprising the amino acids sequence (SEQ ID NO:29).
Tubulin dimers purified from any eukaryotic cells.
Isolated or recombinant peptides from 4 to 50 amino acid residues, in particular 5 to 20 amino acids, preferably 6 to 12 amino acid residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, preferably a tyrosine (Y), in particular comprising an amino acid sequence selected from SEQ ID NO: 33 to SEQ ID NO: 39 or variant thereof.

Preparation of Recombinant TCPases (VASH1, VASH2, CRMP1):

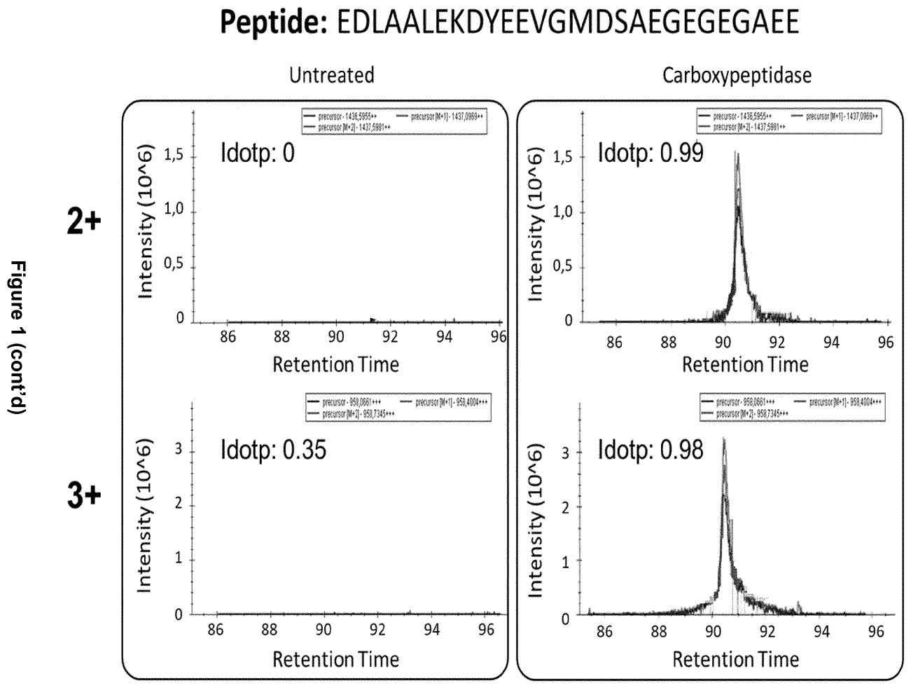

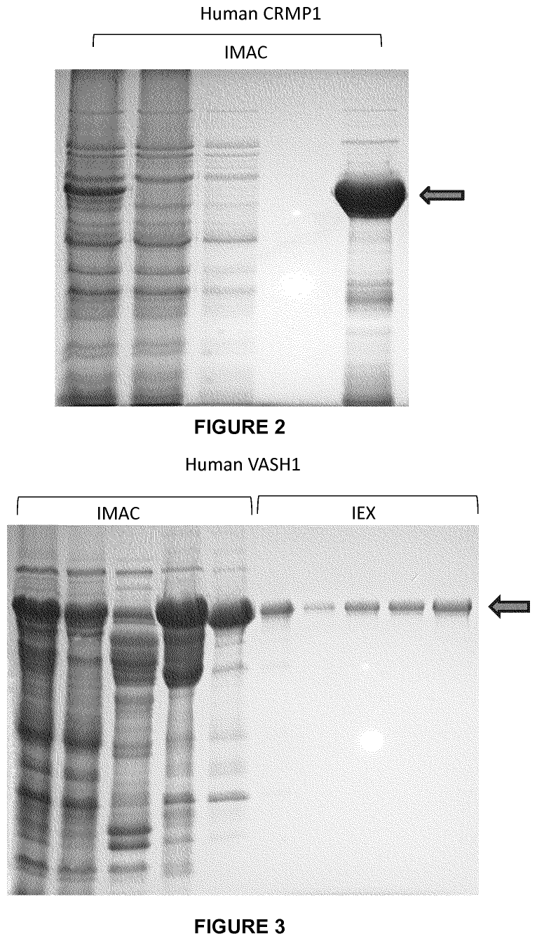

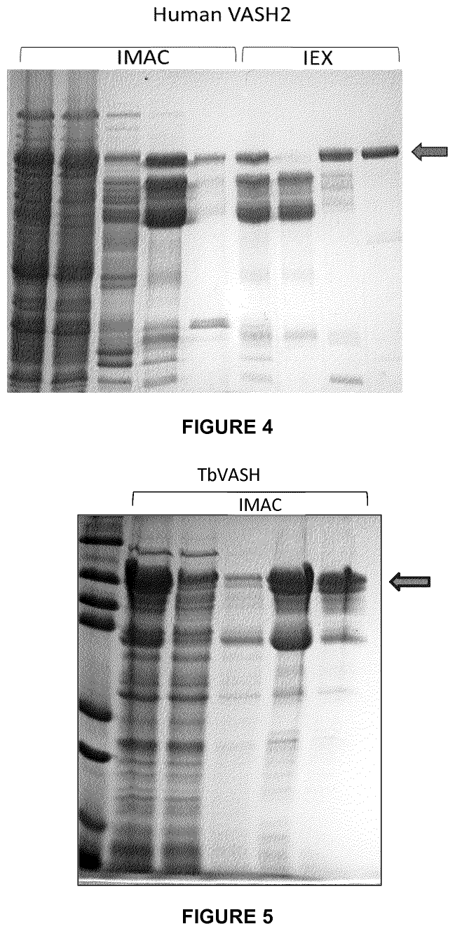

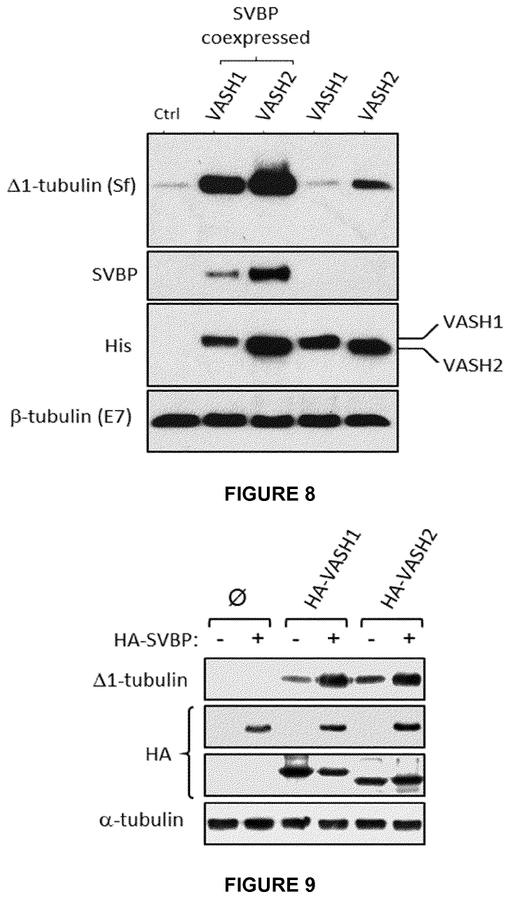

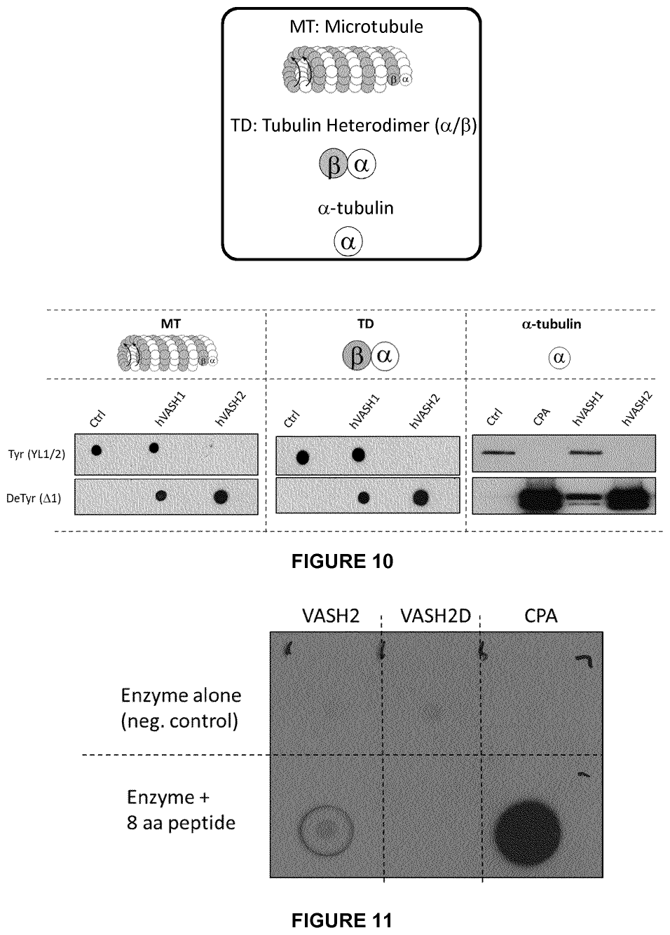

Enzymes are produced in E. coli bacteria using expression vectors such as pET-28a(+), pGEX or any other adapted expression vectors. The different enzymes (human VASH1, VASH2, CRMP1, Trypanosoma brucei VASH) have been cloned and bacteria have been induced according to manufacturer's guidelines. Bacteria were grown until OD600 reached between 0.4-0.8, quickly cooled in ice water for 30 minutes. Protein production was induced using IPTG and incubated at 15° C. for 24 hours under rigorous shaking. The bacteria were pelleted by centrifugation and stored at −80° C. until further use. Bacteria were resuspended in disruption buffer and mechanically disrupted using a French press. Recombinant protein was purified by immobilized metal affinity chromatography (IMAC) and if needed purity was further increased by ion-exchange column (FIG. 2-5). The recombinant proteins have been assessed for their TCPase activity on various substrates. Importantly we found that VASH2 acted as an autonomous enzyme that did not require the small vasohibin binding protein as a chaperone to enzymatically be active (FIG. 8). Advantageously, recombinant purified VASH2 protein could be stored at 4° C. for at least 3 months without noticeable loss of activity. More advantageously, recombinant VASH2 protein showed detyrosination activity on polymerized microtubule, tubulin dimers, recombinant alpha tubulin, engineered Telokin protein containing C-terminal sequence of alpha tubulin and on synthetic peptides (FIG. 10). As such recombinant VASH2 presented unexpected advantageous characteristics allowing its use in various applications such as HTS assays for identification of detyrosinase modulating compounds.

Compounds to be Tested (Putative Modulators of TCPase)

The compounds to be tested may come from chemical libraries of compounds containing a large number of diverse and different compounds. Many of these libraries may be commercially available. Alternatively, smaller libraries may be tested on different substrates (microtubule versus tubulin dimer).

Reagents and Antibodies

For western blot or immunoblot, all antibodies were applied overnight at 4° C. in phosphate-buffered saline (PBS) containing 1% BSA and 0.1% Tween (Sigma). The antibodies used were directed against: Δ1-tubulin (Abcam)), GFP (Millipore), VASH2 (Millipore), beta-tubulin (E7-DSHB), SVBP (Sigma), YL1/2 (Synaptic Systems). The antibody directed against Sf9 Δ1-tubulin (deTyr-tub) were raised in rabbits against the peptide EGEGAEE (=SEQ ID NO: 40).

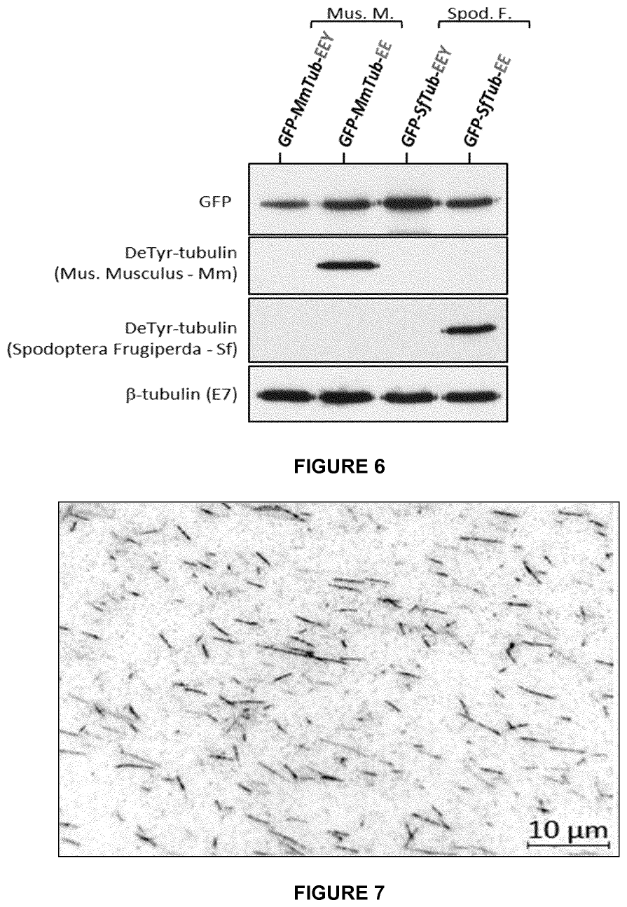

Specific antibodies directed against detyrosinated tubulin were generated. The antibody was raised by injecting rabbits with a peptide composed of the very C-terminal sequence of Spodoptera frugiperda (SF) tubulin. The sequence of the peptide contains the 7 most C-terminal sequence of the protein without the most C-terminal Y (DeTyr) and an additional cysteine at the N-terminal for practical reasons (Seq: C-EGEGAEE=(=SEQ ID NO: 40). Characterization of the antibody was performed by expression of full-length mouse or Spodoptera frugiperda alpha-tubulin or detyrosinated counterpart in HEK cells (FIG. 6).

Advantageously, the said antibody is labelled, ie coupled with a peroxidase, such as the horseradish peroxidase (HRP), and the chromogenic substrate is 3,3′,5,5′-tetramethylbenzidine TMB. For the colorimetric assay the tyrosinase enzyme was purchased at Sigma-Aldrich (Tyrosinase from mushroom) and spectrophotometric analysis was performed using Nanodrop (Thermo Fisher).

Mass Spectrometry

Proteins were separated on SDS-PAGE gels (10% polyacrylamide; Mini-Protean TGX Precast Gels; Bio-Rad) and stained with Page Blue Stain (Fermentas). Gel lanes were cut into several gel pieces and destained by three washes in 50% acetonitrile and 50 mM triethylammonium bicarbonate. Proteins were in-gel digested using trypsin (0.5 μg/band; Gold; Promega).

Peptides were then analyzed online by nano-flow HPLC-nanoelectrospray ionization using a Qexactive Plus mass spectrometer (Thermo Fisher Scientific) coupled to a nano-LC system (U3000-RSLC, Thermo Fisher Scientific). Desalting and preconcentration of samples were performed on-line on a Pepmap® precolumn (0.3×10 mm; Dionex). A gradient consisting of 0-40% B in A for 100 min (A: 0.1% formic acid, 2% acetonitrile in water, and B: 0.1% formic acid in 80% acetonitrile) at 300 nl/min, was used to elute peptides from the capillary reverse-phase column (0.075×150 mm, Pepmap®, Dionex). Data were acquired using the Xcalibur software (version 4.0). A cycle of one full-scan mass spectrum (375-1,500 m/z) at a resolution of 70,000 (at 200 m/z), followed by 12 data-dependent MS/MS spectra (at a resolution of 17,500, isolation window 1.2 m/z) was repeated continuously throughout the nanoLC separation.

Raw data analysis was performed using the MaxQuant software (version 1.5.5.1) with standard settings. Used database consist of Spodoptera frugiperda entries from Uniprot, G3CKA7 sequence (Uniprot) deleted from 1 to 6 amino acids in Cterm and 250 classical contaminants (MaxQuant contaminant database). Relative abundance of peptide was estimated using Skyline 3.6.0.

E. coli Bacterial and Sf Insect Cells Culture and Protein Production (VASH1, VASH2 and SVBP)

Both bacterial and insect cells (Sf9 and Sf21) were used for recombinant protein production. Insect cells Sf9 and Sf21 cells were grown in EX-CELL® 420 Serum-free medium (14420C, Sigma-Aldrich). Cells were maintained between 1 and 10 million cells/ml in shake flasks, at 28 degrees and 140 rpm agitation. For production of human VASH1, VASH2 and SVBP cDNAs were cloned into pFastBac vector MAX efficiency DH10Bac™ Next, competent bacteria (10361-012, Invitrogen) were transformed to produce bacmids. To generate baculoviruses, 12 million Sf21 cells (1 ml) were transfected with bacmid using Cellfectin™ (P/N 58760, Invitrogen). After 5 hours incubation, medium was added and cultures were incubated for 2.5 days. Supernatant (P1) was collected by centrifugation and used to infect 200 million Sf21 cells. Cells were incubated for 2 days and supernatant (P2) was collected by centrifugation. For infection, 40 million Sf21 cells (in 20 ml) were infected with combinations of P2 encoding VASH1, VASH2 and SVBP at 1:50 dilution. Coexpression of VASH1 and VASH2 with SVBP was performed by mixing 1:1 with respective P2 supernatants. After 48 hours, infected cells were collected by centrifugation and analyzed by immunoblotting. For bacterial production of recombinant protein, competent E. coli bacterial cells were transformed with plasmid expression vector containing the protein of interest (VASH1, VASH2, human alpha tubulin, engineered telokin, CRMP1, etc.) and grown on antibiotic containing LB medium. Bacteria were allowed to grow at 37° C. and a single colony was picked. A starter was allowed to grow to confluence in a small LB volume containing the antibiotic overnight. Next, the E. coli bacterial cells were inoculated in appropriate volume and OD600 was monitored regularly using a spectrometer. Once E. coli bacterial cell confluency reached between 0.4 and 0.8 at OD600, bacteria were IPTG induced at 16° C. for 24 hours and collected by centrifugation. Cell pellets were stored at −80° C. until further processed. Purification of recombinant protein was performed using IMAC. The purity of all recombinant proteins were assessed by Coomassie staining.

### Example 1: In Vitro Immunoassay (Immunoblot and ELISA) for Identification of Detyrosinase (TCPase) Modulators Compounds

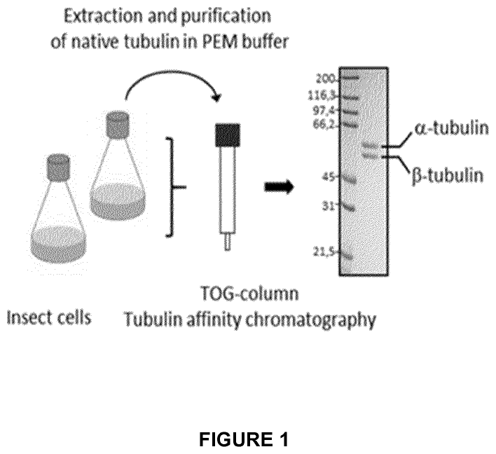

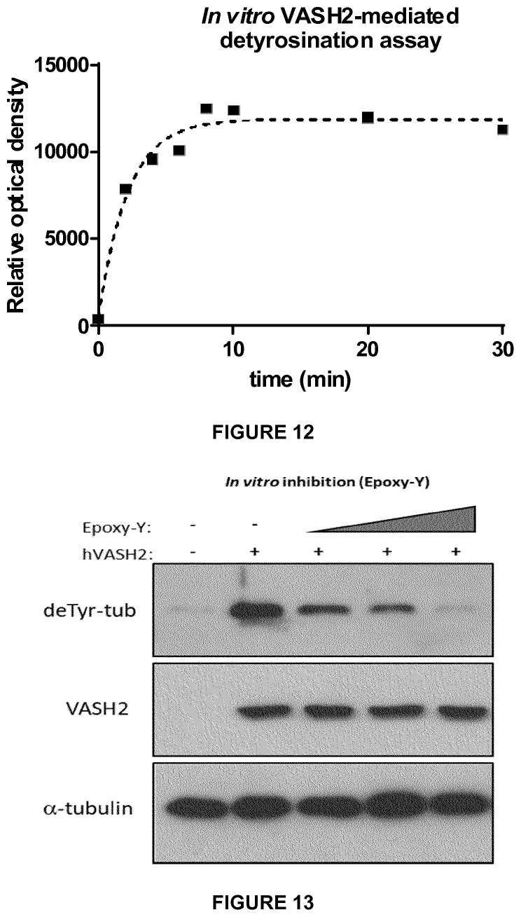

The proof of principle of an immunoassay such as ELISA assay has been obtained by using VASH2 recombinant protein for enzymatic description (FIG. 12). Advantageously the assay has been used in a dose-response analysis using a previously described Epoxy-Y inhibitor (Science 2017, Aillaud et al.). As expected increasing amounts of Epoxy-Y inhibitor resulted in a dose dependent inhibition of VASH2 detyrosinase activity. Oppositely SVBP induces tubulin carboxypeptidase activity of VASH2 (FIG. 9). Next, Tubulin purified from Sf9 insect cells (fully tyrosinated; FIG. 1) was polymerized in presence of Taxol at 37° C. for 30 min, washed and stored in −80° C. until further use. Recombinant VASH2 protein was pre-incubated 5 min at room temperature in presence or absence of Epoxy-Y. The previously prepared microtubules (MTs) (substrate of TCPase, ie VASH2 protein) were added to the reaction mixture in a phosphate buffer containing 100 mM NaCl at pH7.0. The reaction was performed for 10 minutes at room temperature, stopped using sample buffer and analyzed by immunoblotting using specific labelled antibodies directed against Sf9 tubulin (FIG. 13).

The same protocol is further reproduced with the compounds to be tested (putative modulators of TCPase, ie VASH2) in replacement to Epoxy-Y, previously used to validate the assay. So recombinant VASH2 protein was pre-incubated 5 min at room temperature in presence or absence of the compound to be tested. The previously prepared microtubules (MTs) (substrate of TCPase, ie VASH2 protein) were added to the reaction mixture in a phosphate buffer containing 100 mM NaCl at pH7.0. The reaction was performed for 10 minutes at room temperature, stopped using sample buffer and analyzed by immunoblotting using specific labelled antibodies directed against detyrosinated Sf9 tubulin (dTyr-tub). If the signal level (detyrosination) is lower than the control (in absence of the compound to be tested), it means that the compound to be tested is an inhibitor of VASH2, as Epoxy-Y, whereas if the signal level (detyrosination) is higher than the control (in absence of the compound to be tested), it means that the compound to be tested is an activator of VASH2. The man skilled in the art will adapt the concentration of enzyme, substrate and compounds to be tested, in order to favor the detyrosination of substrate (microtubules MT). In an alternative, other substrates of VASH2 are used instead of micrcotubules MT, in particular the ones disclosed in Material & Methods above, and preferably a recombinant tyrosinated telokin such as the one comprising the amino acids sequence SEQ ID NO: 17.

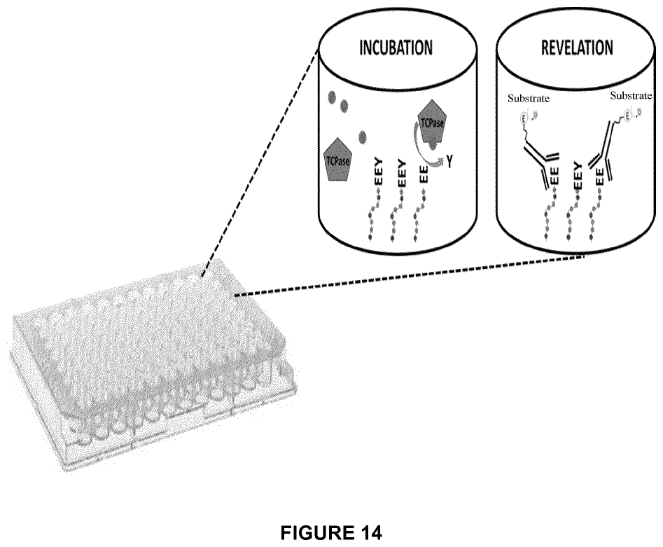

The method then allowed the development of an ELISA-based assay screening of chemical compounds (putative modulator compounds) in a microtiter plate. A schematic representation of an ELISA-based assay for HTS assay is shown in FIG. 14.

The substrate of TCPase is selected from the ones disclosed above in Material & Methods. In particular, we used a recombinant tyrosinated telokin comprising the sequence SEQ ID NO:17 (Mus musculus, fully tyrosinated variant).

As negative control, we used an isolated (non-tyrosinated) telokin comprising the sequence SEQ ID NO:16 Uniprot Q6PDN3-4) without the ultimate Y.

We use the reagents and instructions of commercial ELISA kit.

Standardized amount of recombinant Telokin protein containing the C-terminal sequence of alpha-tubulin as disclosed above is coated to a multiwell plate (96/384/1536).

The substrate coated 96 wells plate is first brought into contact, in absence (control) or presence of a compound to be tested (putative modulator of TCPase), with the recombinant active bacterially produced VASH2 for 15 minutes. The enzyme is washed off using appropriate buffer (preferred is phosphate buffer), and HRP-coupled antibody specifically targeting detyrosinated α-tubulin SF9 (dTyr-Ab SF9), is brought in contact with substrate in each well and excess appropriately washed off with a mild detergent solution to remove antibodies that are non-specifically bound. After the final wash step, the plate is developed by adding Chromogenic HRP substrate TMB is added and microplate measurement is performed accordingly.

### Example 2: In Vitro Colorimetric Assay (Spectrophotometric Assay) for Identification of Detyrosinase (TCPase) Modulators Compounds

The assay relies on the well-document tyrosinase activity on free tyrosine. Free tyrosine can be converted by tyrosinase into a colorimetric detectable compound. This reaction has been described for almost a century (Lichtman, J B C 1929) and applied to measure free tyrosine in various samples.

Tyrosinase catalyzes tyrosine to DOPO and DOPA to DOPAquinone subsequently. During DOPAquinone formation, protons (H+) are produced and these protons could be monitored by substrate (ex: thymol blue) color change (Park et al., 2003).

Current method relies on the use of VASH2 and appropriate substrate of TCPase as disclosed in the above Material & Methods to release free tyrosine in the reaction mixture that can in a second step be used for tyrosinase-dependent analysis and spectrophotometric quantification.

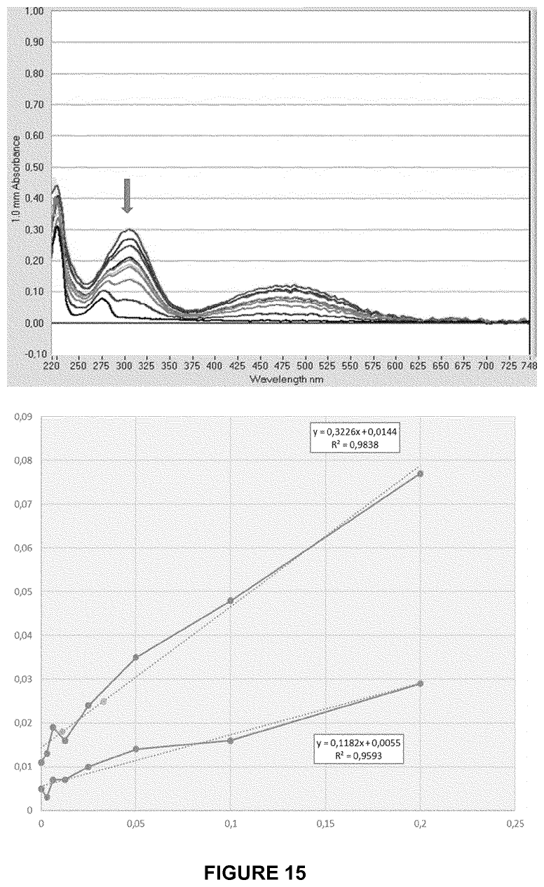

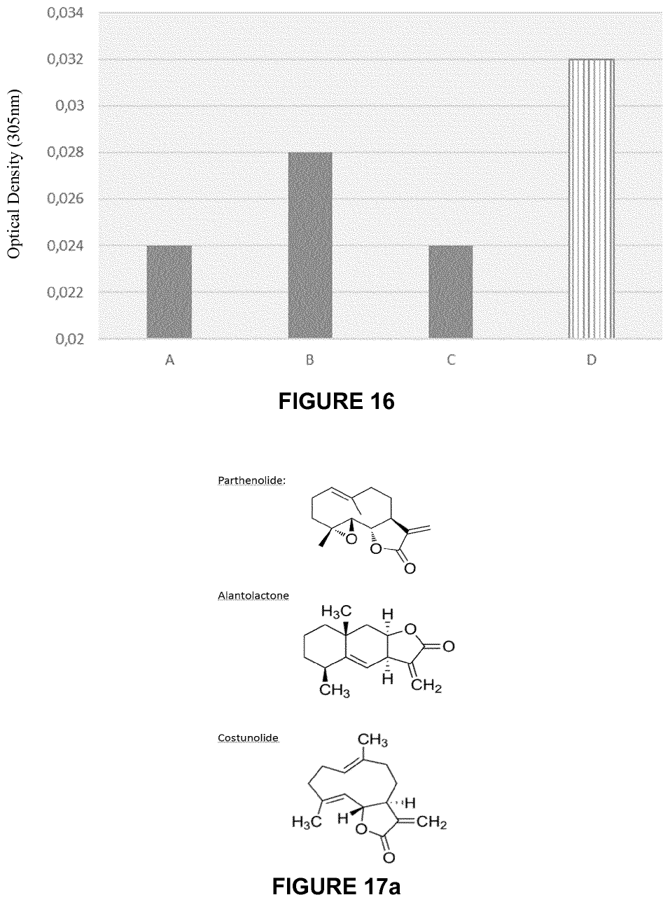

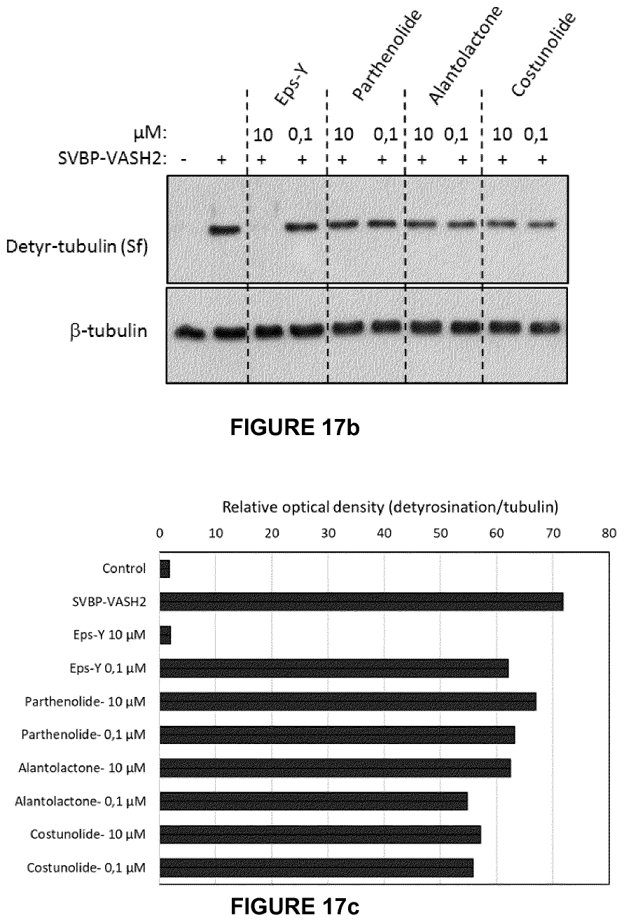

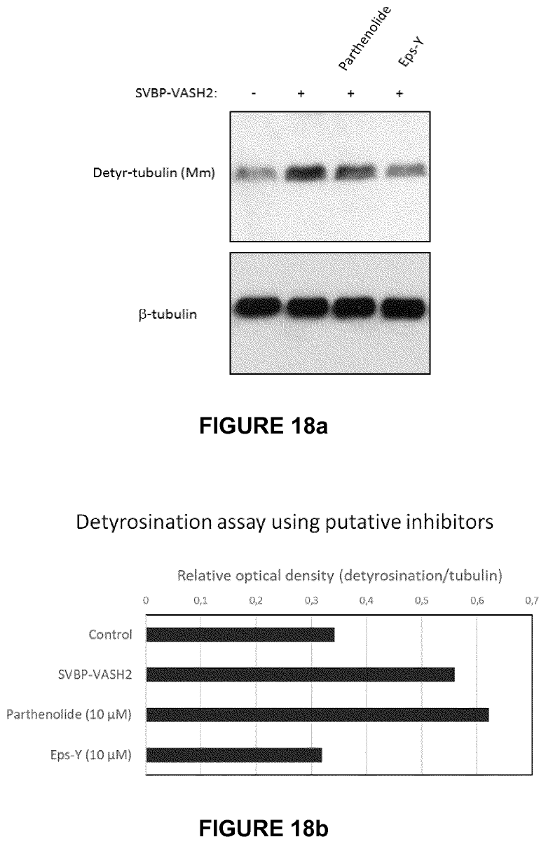

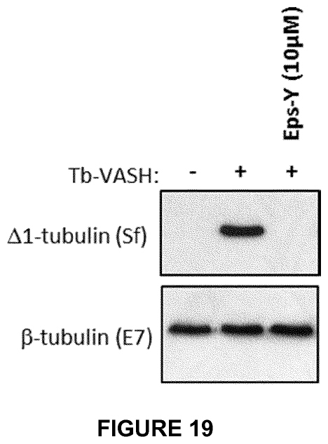

A reaction mixture was prepared by pipetting a phosphate buffer, deionized water, VASH2 recombinant protein, TCPase substrate such as MTs or human alpha tubulin and tyrosinase. The TCPase activity is directly correlated to the increase in absorption due to tyrosinase-mediated generation of dopaquinone. The absorption may be measured with a SPECTRAmax PLUS spectrophotometer. The released C-terminal tyrosine residue by VASH2 becomes substrate of tyrosinase that catalyzes a colorimetric compound that can be measured using a spectrophotometer. We used recombinant tyrosinase (Sigma Aldrich) to determine the reaction kinetics by measuring optical density at 305 nm (FIG. 15). Increasing amounts of free tyrosine were mixed with recombinant tyrosinase in a phosphate buffer pH=7.0, incubated at room temperature for 15 min and analyzed by spectrophotometer at 305 nm (FIG. 15). Next, the proof of concept was performed using recombinant VASH2. Recombinant VASH2 was mixed with bacterially produced recombinant human alpha tubulin, in particular recombinant tubulin alpha-1A chain comprising the amino acid sequence SEQ ID NO: 19 (Uniprot Q71U36) and incubated for 15 min at room temperature in a phosphate buffer ph=7.0, 100 mM NaCl. Next, tyrosinase enzyme was added, mixed and incubated at room temperature for 30 min and analyzed using a spectrophotometer at 305 nm (FIG. 16).

All controls containing the individual components of the reaction were measured (samples A-C) as well.
A. control sample containing tyrosinase,
B. control sample containing recombinant human alpha tubulin,
C. control samples containing tyrosinase (Tyr) and VASH2 as detyrosinase,
D reaction mixture containing recombinant human alpha tubulin, VASH2 mediating tyrosine release and tyrosinase to allow detection of free tyrosine.
OD305 was measured 30 min after onset of reaction at room temperature.
Release of tyrosine by VASH2 could be observed (Sample D—FIG. 16.).

### Example 3: Dot-Blot Assay for Identification of Detyrosinase (TCPase) Modulators Compounds

In this technique, 2 μl of the reaction mixture containing the substrate (8 amino acid peptide consisting of GEEEGEEY) and the enzyme (Recombinant human VASH2) in absence or in presence of the compound were doted on nitrocellulose and assessed for detyrosination of the peptide using a specific antibody detecting detyrosination.

The filter is allowed to dry and the membrane is incubated in blocking buffer to prevent non-specific binding. Similarly to ELISA, HRP-coupled antibody directed against the detyrosinated Telokin engineered substrate (as disclosed above) is incubated over the membrane, excess is washed-off and the membrane is developed using e.g. chromogenic HRP substrate. Intensities of the signals on autoradiograms were quantified by densitometric scanning. Alternatively, we designed a method using a slot blot approach.

The steps are identical as for the dot blot approach, this is the samples are applied to a nitrocellulose or PVDF membrane with a vacuum manifold to produce an orderly grid of samples. Once dry, dot blots and slot blots are subjected to the same immunodetection steps used for Western blotting (FIG. 11).

### Example 4: Detyrosination Assay Performed Using Tubulin Purified from Sf9 Cells and Recombinant SVBP-VASH2 Complex in Absence or Presence of Putative Inhibitors of Detyrosinase

In order to test previously identified compounds as inhibitors of tubulin detyrosinase (Fonrose et al. 2007), we selected the three most promising compounds (Parthenolide, Alantolactone and Costunolide) (FIG. 17a) and assayed their activity in the in vitro detyrosination assay of the present invention using Sf9 tubulin. Using SVBP-VASH2 complex as detyrosinase, two different concentrations (0.1 μM and 10 μM) of these compounds together with Eps-Y (Epoxy Y) were incubated for 15 min at 37° C. in presence of microtubules polymerised using Sf9 cells purified tubulin (substrate of detyrosinase). A negative control sample consisting of the tubulin in the reaction buffer in absence of the detyrosinase (SVBP-VASH2 complex) was also analysed by western blotting (FIG. 17b). Surprisingly, while SVBP-VASH2 complex strongly induced detyrosination in the assay, the in cellulo identified putative inhibitors of detyrosinase including parthenolide, alantolactone and costunolide in human HEK293 cells, did not result in any significant reduction of the detyrosinating activity (FIG. 17c). This suggests that the effect of these compounds in human cells on the level of detyrosination likely reflects an indirect adaptive mechanism that alters microtubule detyrosination more than direct binding and inhibition of the VASH detyrosinating enzymes.

### Example 5: Detyrosination Assay Performed Using Human Tubulin Purified from HEK293 Cells and Recombinant SVBP-VASH2 Complex

Human HEK293 cells were lysed and used for detyrosination assay in presence of parthenolide and Eps-Y compounds. Briefly, HEK293 cells were grown and harvested for further testing the detyrosinating activity of purified recombinant SVBP-VASH2 complex on human microtubules. Using SVBP-VASH2 complex as detyrosinase, extracted human tubulin from HEK293 cells and two different compounds, we performed a detyrosination assay. Samples were incubated for 15 min at 37° C. and analysed by western blot (FIG. 18a). Consistent with previous results and as opposed to Eps-Y, parthenolide did not result in inhibition of detyrosination activity in this assay (FIG. 18b). Moreover, the fold induction of the tubulin detyrosination resulting from SVBP-VASH2 complex was significantly lower with human tubulin purified from HEK293 cells in comparison to the one obtained using Sf9 cells extracted tubulin. This is likely to be the result of the numerous modifications tubulin is exposed to in human cells. The surprising observation that Sf cells extracted tubulin do not bare significant amount of tubulin modification is further exemplified by this observation. As expected, Eps-Y reduced detyrosination to background levels.

These results showed that the Sf cells extracted tubulin is of particular interest, as substrate of TCPase, in the in vitro assay of the present invention, because it does not bare significant amount of tubulin modification.

### Example 6: Detvrosination Assay Performed Using Sf Tubulin and Recombinant Trypanosoma brucei VASH Protein

To test the use of the in vitro detyrosination system to other detyrosinase, we cloned the VASH gene of Trypanosoma brucei, a protozoan parasite that is responsible for neglected tropical diseases such as Human African Trypanosomiasis. Trypanosoma brucei cells were lysed and using a reverse transcription kit (Takara), we produced cDNA. Next, by PCR we specifically amplified the VASH gene and cloned into a bacterial expression vector. Purification protocols were optimized and recombinant Tb-VASH protein (SEQ ID NO: 14 (Uncharacterized protein/VASH of Trypanosoma brucei), was assayed using the in vitro detyrosination assay of the present invention. Using Tb-VASH as detyrosinase, samples were incubated for 15 min at 37° C. in presence of microtubules polymerised using Sf9 cells purified tubulin. A negative control sample consisting of the tubulin in the reaction buffer in absence of the detyrosinase was also analysed by western blotting. As expected, Tb-VASH was efficient in removing the C-terminal Y residue contained by the α-tubulin sequence (FIG. 19). As such, we provided evidence that the assay can be adapted to test any tubulin detyrosinase to identify compounds that directly modulate its activity.

### REFERENCES

Aillaud, C., Bosc, C., Peris, L., Bosson, A., Heemeryck, P., Van Dijk, J., Le Friec, J., Boulan, B., Vossier, F., Sanman, L. E., et al. (2017). Vasohibins/SVBP are tubulin carboxypeptidases (TCPs) that regulate neuron differentiation. Science 358, 1448-1453 Ciferri, C., Pasqualato, S., Screpanti, E., Varetti, G., Santaguida, S., Dos Reis, G., Maiolica, A., Polka, J., De Luca, J. G., De Wulf, P., et al. (2008). Implications for kinetochore-microtubule attachment from the structure of an engineered Ndc80 complex. Cell 133, 427-439.
Fonrose et al., (2007). “Parthenolide inhibits Tubulin Carboxypeptidase Activity”, Cancer research, vol. 67, n° 7, 1 Apr. 2007, pages 3371-3378.
Gordon, Malcolm and Mitchell, Herschel K. (1949) A fluorometric method for the estimation of tryptophan. Journal of Biological Chemistry, 180 (3). pp. 1065-1070. ISSN 0021-9258.
Inglese J, et al. Nat Chem Biol. 2007 August; 3(8):466-79. Review
Kato et al., 2004 (Int J Cancer), November 10; 112(3):365-75. Low expression of human tubulin tyrosine ligase and suppressed tubulin tyrosination/detyrosination cycle are associated with impaired neuronal differentiation in neuroblastomas with poor prognosis.
Lichtman, SS, Sobotka H. J. Biol. Chem 1929, 85:261-273
Magiera M M, Singh P, Gadadhar S, Janke C. Cell. 2018 May 31; 173(6):1323-1327. doi: 10.1016/j.cell.2018.05.018. Epub 2018 May 31. Review. Tubulin Posttranslational Modifications and Emerging Links to Human Disease.
Barisic M, Silva e Sousa R, Tripathy S K, Magiera M M, Zaytsev A V, Pereira A L, Janke C, Grishchuk E L, Maiato H. Science. 2015 May 15; 348(6236):799-803. Mialhe et al., 2001 (Cancer Res), 2001 Jul. 1; 61(13):5024-7. Tubulin detyrosination is a frequent occurrence in breast cancers of poor prognosis.
Nieuwenhuis, J., Adamopoulos, A., Bleijerveld, O. B., Mazouzi, A., Stickel, E., Celie, P., Altelaar, M., Knipscheer, P., Perrakis, A., Blomen, V. A., et al. (2017). Vasohibins encode tubulin detyrosinating activity. Science 358, 1453-1456.
Park et al. 2003, Journal of Protein Chemistry, Vol. 22, No 5, 473-480. A new continuous spectrophotometric assay method for DOPA oxidase activity of tyrosinase.
Preston, S. F., Deanin, G. G., Hanson, R. K., and Gordon, M. W. (1979). The phylogenetic distribution of tubulin:tyrosine ligase. J Mol Evol 13, 233-244.
Roll-Mecak, A., and Vale, R. D. (2008). Structural basis of microtubule severing by the hereditary spastic paraplegia protein spastin. Nature 451, 363-367.
Widlund, P. O., Podolski, M., Reber, S., Alper, J., Storch, M., Hyman, A. A., Howard, J., and Drechsel, D. N. (2012). One-step purification of assembly-competent tubulin from diverse eukaryotic sources. Mol Biol Cell 23, 4393-4401.

1.-15. (canceled)

16. An in vitro screening assay for identification of modulators of tubulin carboxypeptidase (TCPase) activity comprising the steps of:
(i) contacting (a) a substrate of TCPase enzyme comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, and (b) an isolated or recombinant TCPase enzyme; in the presence or absence (control) of the modulator compound to be tested, and under conditions for substrate cleavage, and/or liberation of a C-terminal free aromatic amino acid residue;
(ii) using reagents for detecting and measuring the signal related to substrate cleavage, and/or liberation of the C-terminal free aromatic amino acid residue;
(iii) measuring and comparing the level of substrate cleavage, and/or the level of the C-terminal free aromatic amino acid residue, in the presence and in the absence (control) of the compound to be tested, and
(iv) identifying the modulators of TCPase for which the level of substrate cleavage, or liberation of the C-terminal free aromatic amino acid residue, is increased in the presence of the compound to be tested (activators of TCPase) or decreased in the presence of the compound to be tested (inhibitors of TCPase).

17. The in vitro screening assay of claim 16, wherein the substrate of TCPase enzyme comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue, is selected from the group consisting of:
a) purified or recombinant α-tubulin,
b) recombinant engineered telokin,
c) isolated or recombinant peptide from 4 to 50 amino acid residues comprising an amino acid sequence having at least the last 4 amino acids residues of the C-terminal sequence of an α-tubulin and/or a Microtubule Associated Protein (MAP) and, as ultimate C-terminal amino acid residue, an aromatic amino acid residue,
d) isolated or recombinant microtubules,
e) isolated or recombinant tubulin dimers of alpha and beta tubulin, f) microtubule-associated proteins (MAP), and
g) mixtures thereof.

18. The in vitro screening assay according to claim 16, wherein the substrate of TCPase enzyme is selected from recombinant engineered telokin having at least the last 4 amino acids residues of the C-terminal sequence of an α-tubulin and a tyrosine (Y) as ultimate C-terminal amino acid residue.

19. The in vitro screening assay according to claim 16, wherein the isolated or recombinant TCPase enzyme is selected from proteins having at least 30%, 40%, 50%, 60%, 70%, 80%, 90%, 95%, or 99% amino acid sequence identity with an amino acid sequence selected from the group consisting of SEQ ID NO:1 (Ubiquitin carboxyl-terminal hydrolase 14), SEQ ID NO:2 (Ubiquitin carboxyl-terminal hydrolase 5), SEQ ID NO:3 (Methionine aminopeptidase 2), SEQ ID NO:4 (Xaa-Pro aminopeptidase 1), SEQ ID NO:5 (Tripeptidyl-peptidase 2), SEQ ID NO:6 (Vasohibin-1), SEQ ID NO:7 (Dihydropyrimidinase-related protein 1, CRM P1), SEQ ID NO:8 (Dihydropyrimidinase-related protein 2), SEQ ID NO:9 (Dihydropyrimidinase-related protein 3), SEQ ID NO:10 (Dihydropyrimidinase-related protein 4), SEQ ID N0:11 (Dihydropyrimidinase-related protein 5), SEQ ID NO:12 (Vasohibin-2), and SEQ ID NO:14 (Uncharacterized protein/VASH of Trypanosoma bruce;), in the presence or absence of SEQ ID NO:13 (Small vasohibin-binding protein).

20. The in vitro screening assay according to claim 19, wherein (a) the substrate of TCPase enzyme is selected from purified or recombinant α-tubulin from parasites, and (b) the isolated or recombinant TCPase enzyme is selected from parasites proteins.

21. The in vitro screening assay according to claim 19, wherein (a) the substrate of TCPase enzyme is a recombinant engineered telokin, and (b) the isolated or recombinant TCPase enzyme is selected from proteins having at least 30%, 40%, 50%, 60%, 70%, 80%, 90%, 95%, or 99% amino acid sequence identity with amino acid sequence SEQ ID NO:6 (Vasohibin-1) or SEQ ID NO:12 (Vasohibin-2) in the presence or absence of SEQ ID NO:13 (Small vasohibin-binding protein).

22. The in vitro screening assay according to claim 16, wherein step (ii) comprises detection of the C-terminal free aromatic amino-acid via a colorimetric assay or using fluorescent properties of the aromatic residues.

23. The in vitro screening assay according to claim 16, wherein the assay is an immuno-assay, in which the step (ii) comprises:
adding an effective amount of specific labelled antibody raised against cleaved substrate, under conditions that favor the formation of a complex antibody-cleaved substrate; and
means for revealing the labelled signal;
or, alternatively,
adding an effective amount of a primary specific antibody raised against cleaved substrate, under conditions that favor the formation of a complex antibody-cleaved substrate;
adding an effective amount of a secondary labelled antibody specific of the primary antibody, under conditions that favor the formation of a complex primary antibody-cleaved substrate-labelled secondary antibody, and means for revealing the labelled signal,
wherein the reaction occurs in soluble (fluid) phase or solid phase.

24. The in vitro screening assay according to claim 23, wherein the antibody is labelled with a marker selected in the group consisting of an enzyme, a fluorescent compound or fluorophore, a (chemo)luminescent compound, and a radioactive element.

25. The in vitro screening assay according to claim 23, wherein said immunoassay is an enzyme immunoassay, a fluoroimmunoassay, a luminescent immunoassay, or a radioimmunoassay.

26. The in vitro screening immunoassay according to claim 23, wherein the reaction occurs in solid phase and wherein:
substrate of TCPase is coated on solid support and the TCPase enzyme, the compound(s) to be tested, and the antibodies raised against the cleaved substrate are added in the reaction solution, or
the antibodies raised against cleaved substrate are coated on a solid support, and the TCPase enzyme, substrate of TCPase, the compound(s) to be tested, and secondary labelled antibodies raised against the cleaved substrate are added in the reaction solution, or
the TCPase enzyme is coated on a solid support and the compound(s) to be tested and the antibodies raised against the cleaved substrate are added in the reaction solution.

27. The in vitro screening immuno-assay according to claim 23, wherein (a) the substrate of TCPase comprises dimers of alpha-tubulin extracted from SF9 cells (Spodoptera frugiperda origin), (b) the TCPase enzyme is vasohibin-2 (VASH2) in the presence or absence of SVBP, and the step (ii) of detecting and measuring the level of detyrosinated-substrate comprises at least an antibody raised against detyrosinated α-tubulin SF9 (dTyr-Ab SF9) coupled to horseradish peroxidase (HRP).

28. The in vitro screening assay according to claim 16, which is a High Throughput Screening (HTS) assay.

29. A kit for performing the in vitro screening immuno-assay of claim 23, comprising:
(i) a TCPase enzyme substrate, and, as ultimate C-terminal amino acid residue, an aromatic amino acid residue,
(ii) a TCPase enzyme,
(iii) antibodies raised against cleaved substrate,
(iv) a negative control,
(v) a positive control,
(vi) a fluid vessel or a solid support for coating or pre-coating either the substrate of TCPase or the TCPase enzyme,
(vii) reagents for allowing contact of said substrate with TCPase enzyme in reaction conditions for substrate cleavage,
(viii) reagents for detecting and measuring the level of substrate cleavage; and
(ix) optionally, instructions for use.

30. A kit for performing the in vitro screening assay of claim 22 for detection of the free C-terminal aromatic amino-acid via a colorimetric assay or using fluorescent properties of the aromatic residues, comprising:
(i) a TCPase enzyme substrate, and, as ultimate C-terminal amino acid residue, an aromatic amino acid residue,
(ii) a TCPase enzyme,
(iii) optionally, a tyrosinase for the detection of a C-terminal free-tyrosine via colorimetric assay,
(iv) a fluid vessel or a solid support for coating or pre-coating either the substrate of TCPase or the TCPase enzyme,
(v) reagents for allowing contact of said substrate with TCPase enzyme in reaction conditions for liberation of a C-terminal free-aromatic amino acid residue,
(vi) reagents for detecting and measuring the level of the C-terminal free-aromatic amino acid residue; and
(vii) optionally, instructions for use.

31. The in vitro screening assay of claim 16, wherein said substrate cleavage is detyrosination and said C-terminal free aromatic amino acid residue is a C-terminal free tyrosine.

32. The in vitro screening assay of claim 17, wherein:
(a) said aromatic amino acid residue is a tyrosine (Y),
(b) said purified or recombinant α-tubulin comprises an amino acid sequence selected from the group consisting of: SEQ ID NO:19 to SEQ ID NO: 24 (Homo sapiens); SEQ ID NO:25 (Trypanosoma bruce;); SEQ ID NO:26 (Trypanosoma bruce;); SEQ ID NO:27 (Toxoplasma gondii); SEQ ID NO:28 (Spodoptera frugiperda); SEQ ID NO:29 (Drosophila melanogaster); and variants thereof,
(c) said recombinant engineered telokin comprises the amino acid sequence of SEQ ID NO:17 or a variant thereof,
(d) said isolated or recombinant peptide has 6 to 12 amino acid residues, and
(e) said microtubule-associated protein is one from RP/EB family members 1-3.

33. The in vitro screening immunoassay of claim 26, wherein:
(a) said solid support is a membrane or microplate, and
(b) said antibodies raised against cleaved substrate are detyrosinated α-tubulin (dTyr-Ab), optionally labelled or combined with labelled secondary antibodies raised against primary antibodies.

34. The kit according to claim 29, wherein:
(a) said TCPase enzyme substrate is selected from (i1) a purified or recombinant tubulin from Spodoptera Frugiperda (Sf) cells, (i2) a recombinant engineered telokin as the substrate of TCPase enzyme, and (i3) recombinant peptides from 5 to 20 amino acid residues comprising an amino acid sequence having at least the last 4 amino acids residues of the C-terminal sequence of an α-tubulin and/or a Microtubule Associated Protein (MAP),
(b) said aromatic amino acid residue is a tyrosine (Y),
(c) said TCPase enzyme is an isolated or recombinant TCPase enzyme selected from proteins having at least 30%, 40%, 50%, 60%, 70%, 80%, 90%, 95%, or 99% amino acid sequence identity with the amino acid sequence of SEQ ID NO:12 (Vasohibin-2) in the presence or absence of SEQ ID NO:13 (Small vasohibin-binding protein),
(d) said antibodies raised against cleaved substrate are raised against detyrosinated α-tubulin SF9 (dTyr-Ab SF9) and coupled to horseradish peroxidase (HRP),
(e) said negative control is non-tyrosinated isolated or recombinant telokin,
(f) said positive control is one comprising Epoxy-Y compound,
(g) said solid support is a microplate, and
(h) said substrate cleavage is detyrosination.

35. The kit according to claim 30, wherein:
(a) said TCPase enzyme substrate is selected from (i1) a purified or recombinant tubulin from Spodoptera Frugiperda (Sf) cells, (i2) a recombinant engineered telokin as the substrate of TCPase enzyme, and (i3) recombinant peptides from 5 to 20 amino acid residues comprising an amino acid sequence having at least the last 4 amino acids residues of the C-terminal sequence of an α-tubulin and/or a Microtubule Associated Protein (MAP),
(b) said aromatic amino acid residue is a tyrosine (Y),
(c) said TCPase enzyme is an isolated or recombinant TCPase enzyme selected from proteins having at least 30%, 40%, 50%, 60%, 70%, 80%, 90%, 95%, or 99% amino acid sequence identity with the amino acid sequence of SEQ ID NO:12 (Vasohibin-2) in the presence or absence of SEQ ID NO:13 (Small vasohibin-binding protein),
(d) said solid support is a microplate, and
(e) said C-terminal free-aromatic amino acid residue is a C-terminal free-tyrosine.


SEQUENCE LISTING (40 sequences)

SEQ ID NO: 1 - protein, 494 aa, Homo sapiens (Human)
  SITE (1)..(494): Ubiquitin carboxyl-terminal hydrolase 14
MPLYSVTVKWGKEKFEGVELNTDEPPMVFKAQLFALTGVQPARQKVMVKGGTLKDDDWGNIKIKNGMTLLMMGSADALPEEPSAKTVFVEDMTEEQLASAMELPCGLTNLGNTCYMNATVQCIRSVPELKDALKRYAGALRASGEMASAQYITAALRDLFDSMDKTSSSIPPIILLQFLHMAFPQFAEKGEQGQYLQQDANECWIQMMRVLQQKLEAIEDDSVKETDSSSASAATPSKKKSLIDQFFGVEFETTMKCTESEEEEVTKGKENQLQLSCFINQEVKYLFTGLKLRLQEEITKQSPTLQRNALYIKSSKISRLPAYLTIQMVRFFYKEKESVNAKVLKDVKFPLMLDMYELCTPELQEKMVSFRSKFKDLEDKKVNQQPNTSDKKSSPQKEVKYEPFSFADDIGSNNCGYYDLQAVLTHQGRSSSSGHYVSWVKRKQDEWIKFDDDKVSIVTPEDILRLSGGGDWHIAYVLLYGPRRVEIMEEESEQ

SEQ ID NO: 2 - protein, 858 aa, Homo sapiens (Human)
  SITE (1)..(858): Ubiquitin carboxyl-terminal hydrolase 5
MAELSEEALLSVLPTIRVPKAGDRVHKDECAFSFDTPESEGGLYICMNTFLGFGKQYVERHFNKTGQRVYLHLRRTRRPKEEDPATGTGDPPRKKPTRLAIGVEGGFDLSEEKFELDEDVKIVILPDYLEIARDGLGGLPDIVRDRVTSAVEALLSADSASRKQEVQAWDGEVRQVSKHAFSLKQLDNPARIPPCGWKCSKCDMRENLWLNLTDGSILCGRRYFDGSGGNNHAVEHYRETGYPLAVKLGTITPDGADVYSYDEDDMVLDPSLAEHLSHFGIDMLKMQKTDKTMTELEIDMNQRIGEWELIQESGVPLKPLFGPGYTGIRNLGNSCYLNSVVQVLFSIPDFQRKYVDKLEKIFQNAPTDPTQDFSTQVAKLGHGLLSGEYSKPVPESGDGERVPEQKEVQDGIAPRMFKALIGKGHPEFSTNRQQDAQEFFLHLINMVERNCRSSENPNEVFRFLVEEKIKCLATEKVKYTQRVDYIMQLPVPMDAALNKEELLEYEEKKRQAEEEKMALPELVRAQVPFSSCLEAYGAPEQVDDFWSTALQAKSVAVKTTRFASFPDYLVIQIKKFTFGLDWVPKKLDVSIEMPEELDISQLRGTGLQPGEEELPDIAPPLVTPDEPKGSLGFYGNEDEDSFCSPHFSSPTSPMLDESVIIQLVEMGFPMDACRKAVYYTGNSGAEAAMNWVMSHMDDPDFANPLILPGSSGPGSTSAAADPPPEDCVTTIVSMGFSRDQALKALRATNNSLERAVDWIFSHIDDLDAEAAMDISEGRSAADSISESVPVGPKVRDGPGKYQLFAFISHMGTSTMCGHYVCHIKKEGRWVIYNDQKVCASEKPPKDLGYIYFYQRVAS

SEQ ID NO: 3 - protein, 478 aa, Homo sapiens (Human)
  SITE (1)..(478): Methionine aminopeptidase 2
MAGVEEVAASGSHLNGDLDPDDREEGAASTAEEAAKKKRRKKKKSKGPSAAGEQEPDKESGASVDEVARQLERSALEDKERDEDDEDGDGDGDGATGKKKKKKKKKRGPKVQTDPPSVPICDLYPNGVFPKGQECEYPPTQDGRTAAWRTTSEEKKALDQASEEIWNDFREAAEAHRQVRKYVMSWIKPGMTMIEICEKLEDCSRKLIKENGLNAGLAFPTGCSLNNCAAHYTPNAGDTTVLQYDDICKIDFGTHISGRIIDCAFTVTFNPKYDTLLKAVKDATNTGIKCAGIDVRLCDVGEAIQEVMESYEVEIDGKTYQVKPIRNLNGHSIGQYRIHAGKTVPIVKGGEATRMEEGEVYAIETFGSTGKGVVHDDMECSHYMKNFDVGHVPIRLPRTKHLLNVINENFGTLAFCRRWLDRLGESKYLMALKNLCDLGIVDPYPPLCDIKGSYTAQFEHTILLRPTCKEVVSRGDDY

SEQ ID NO: 4 - protein, 623 aa, Homo sapiens (Human)
  SITE (1)..(623): Xaa-Pro aminopeptidase 1
MPPKVTSELLRQLRQAMRNSEYVTEPIQAYIIPSGDAHQSEYIAPCDCRRAFVSGFDGSAGTAIITEEHAAMWTDGRYFLQAAKQMDSNWTLMKMGLKDTPTQEDWLVSVLPEGSRVGVDPLIIPTDYWKKMAKVLRSAGHHLIPVKENLVDKIWTDRPERPCKPLLTLGLDYTGISWKDKVADLRLKMAERNVMWFVVTALDEIAWLFNLRGSDVEHNPVFFSYAIIGLETIMLFIDGDRIDAPSVKEHLLLDLGLEAEYRIQVHPYKSILSELKALCADLSPREKVWVSDKASYAVSETIPKDHRCCMPYTPICIAKAVKNSAESEGMRRAHIKDAVALCELFNWLEKEVPKGGVTEISAADKAEEFRRQQADFVDLSFPTISSTGPNGAIIHYAPVPETNRTLSLDEVYLIDSGAQYKDGTTDVTRTMHFGTPTAYEKECFTYVLKGHIAVSAAVFPTGTKGHLLDSFARSALWDSGLDYLHGTGHGVGSFLNVHEGPCGISYKTFSDEPLEAGMIVTDEPGYYEDGAFGIRIENVVLVVPVKTKYNFNNRGSLTFEPLTLVPIQTKMIDVDSLTDKECDWLNNYHLTCRDVIGKELQKQGRQEALEWLIRETQPISKQH

SEQ ID NO: 5 - protein, 1249 aa, Homo sapiens (Human)
  SITE (1)..(1249): Tripeptidyl-peptidase 2
MATAATEEPFPFHGLLPKKETGAASFLCRYPEYDGRGVLIAVLDTGVDPGAPGMQVTTDGKPKIVDIIDTTGSGDVNTATEVEPKDGEIVGLSGRVLKIPASWTNPSGKYHIGIKNGYDFYPKALKERIQKERKEKIWDPVHRVALAEACRKQEEFDVANNGSSQANKLIKEELQSQVELLNSFEKKYSDPGPVYDCLVWHDGEVWRACIDSNEDGDLSKSTVLRNYKEAQEYGSFGTAEMLNYSVNIYDDGNLLSIVTSGGAHGTHVASIAAGHFPEEPERNGVAPGAQILSIKIGDTRLSTMETGTGLIRAMIEVINHKCDLVNYSYGEATHWPNSGRICEVINEAVWKHNIIYVSSAGNNGPCLSTVGCPGGTTSSVIGVGAYVSPDMMVAEYSLREKLPANQYTWSSRGPSADGALGVSISAPGGAIASVPNWTLRGTQLMNGTSMSSPNACGGIALILSGLKANNIDYTVHSVRRALENTAVKADNIEVFAQGHGIIQVDKAYDYLVQNTSFANKLGFTVTVGNNRGIYLRDPVQVAAPSDHGVGIEPVFPENTENSEKISLQLHLALTSNSSWVQCPSHLELMNQCRHINIRVDPRGLREGLHYTEVCGYDIASPNAGPLFRVPITAVIAAKVNESSHYDLAFTDVHFKPGQIRRHFIEVPEGATWAEVTVCSCSSEVSAKFVLHAVQLVKQRAYRSHEFYKFCSLPEKGTLTEAFPVLGGKAIEFCIARWWASLSDVNIDYTISFHGIVCTAPQLNIHASEGINRFDVQSSLKYEDLAPCITLKNWVQTLRPVSAKTKPLGSRDVLPNNRQLYEMVLTYNFHQPKSGEVTPSCPLLCELLYESEFDSQLWIIFDQNKRQMGSGDAYPHQYSLKLEKGDYTIRLQIRHEQISDLERLKDLPFIVSHRLSNTLSLDIHENHSFALLGKKKSSNLTLPPKYNQPFFVTSLPDDKIPKGAGPGCYLAGSLTLSKTELGKKADVIPVHYYLIPPPTKTKNGSKDKEKDSEKEKDLKEEFTEALRDLKIQWMTKLDSSDIYNELKETYPNYLPLYVARLHQLDAEKERMKRLNEIVDAANAVISHIDQTALAVYIAMKTDPRPDAATIKNDMDKQKSTLVDALCRKGCALADHLLHTQAQDGAISTDAEGKEEEGESPLDSLAETFWETTKWTDLFDNKVLTFAYKHALVNKMYGRGLKFATKLVEEKPTKENWKNCIQLMKLLGWTHCASFTENWLPIMYPPDYCVF

SEQ ID NO: 6 - protein, 365 aa, Homo sapiens (Human)
  SITE (1)..(365): Vasohibin-1
MPGGKKVAGGGSSGATPTSAAATAPSGVRRLETSEGTSAQRDEEPEEEGEEDLRDGGVPFFVNRGGLPVDEATWERMWKHVAKIHPDGEKVAQRIRGATDLPKIPIPSVPTFQPSTPVPERLEAVQRYIRELQYNHTGTQFFEIKKSRPLTGLMDLAKEMTKEALPIKCLEAVILGIYLTNSMPTLERFPISFKTYFSGNYFRHIVLGVNFAGRYGALGMSRREDLMYKPPAFRTLSELVLDFEAAYGRCWHVLKKVKLGQSVSHDPHSVEQIEWKHSVLDVERLGRDDFRKELERHARDMRLKIGKGTGPPSPTKDRKKDVSSPQRAQSSPHRRNSRSERRPSGDKKTSEPKAMPDLNGYQIRV

SEQ ID NO: 7 - protein, 572 aa, Homo sapiens (Human)
  SITE (1)..(572): Dihydropyrimidinase-related protein 1
  SITE (1)..(572): CRMP1
MSYQGKKSIPHITSDRLLIKGGRIINDDQSLYADVYLEDGLIKQIGENLIVPGGVKTIEANGRMVIPGGIDVNTYLQKPSQGMTAADDFFQGTRAALVGGTTMIIDHVVPEPGSSLLTSFEKWHEAADTKSCCDYSLHVDITSWYDGVREELEVLVQDKGVNSFQVYMAYKDVYQMSDSQLYEAFTFLKGLGAVILVHAENGDLIAQEQKRILEMGITGPEGHALSRPEELEAEAVFRAITIAGRINCPVYITKVMSKSAADIIALARKKGPLVFGEPIAASLGTDGTHYWSKNWAKAAAFVTSPPLSPDPTTPDYLTSLLACGDLQVTGSGHCPYSTAQKAVGKDNFTLIPEGVNGIEERMTVVWDKAVATGKMDENQFVAVTSTNAAKIFNLYPRKGRIAVGSDADVVIWDPDKLKTITAKSHKSAVEYNIFEGMECHGSPLVVISQGKIVFEDGNINVNKGMGRFIPRKAFPEHLYQRVKIRNKVFGLQGVSRGMYDGPVYEVPATPKYATPAPSAKSSPSKHQPPPIRNLHQSNFSLSGAQIDDNNPRRTGHRIVAPPGGRSNITSLG

SEQ ID NO: 8 - protein, 572 aa, Homo sapiens (Human)
  SITE (1)..(572): Dihydropyrimidinase-related protein 2
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 9 - protein, 570 aa, Homo sapiens (Human)
  SITE (1)..(570): Dihydropyrimidinase-related protein 3
MSYQGKKNIPRITSDRLLIKGGRIVNDDQSFYADIYMEDGLIKQIGDNLIVPGGVKTIEANGKMVIPGGIDVHTHFQMPYKGMTTVDDFFQGTKAALAGGTTMIIDHVVPEPESSLTEAYEKWREWADGKSCCDYALHVDITHWNDSVKQEVQNLIKDKGVNSFMVYMAYKDLYQVSNTELYEIFTCLGELGAIAQVHAENGDIIAQEQTRMLEMGITGPEGHVLSRPEELEAEAVFRAITIASQTNCPLYVTKVMSKSAADLISQARKKGNVVFGEPITASLGIDGTHYWSKNWAKAAAFVTSPPLSPDPTTPDYINSLLASGDLQLSGSAHCTFSTAQKAIGKDNFTAIPEGTNGVEERMSVIWDKAVATGKMDENQFVAVTSTNAAKIFNLYPRKGRISVGSDSDLVIWDPDAVKIVSAKNHQSAAEYNIFEGMELRGAPLVVICQGKIMLEDGNLHVTQGAGRFIPCSPFSDYVYKRIKARRKMADLHAVPRGMYDGPVFDLTTTPKGGTPAGSARGSPTRPNPPVRNLHQSGFSLSGTQVDEGVRSASKRIVAPPGGRSNITSLS

SEQ ID NO: 10 - protein, 572 aa, Homo sapiens (Human)
  SITE (1)..(572): Dihydropyrimidinase-related protein 4
MSFQGKKSIPRITSDRLLIRGGRIVNDDQSFYADVHVEDGLIKQIGENLIVPGGIKTIDAHGLMVLPGGVDVHTRLQMPVLGMTPADDFCQGTKAALAGGTTMILDHVFPDTGVSLLAAYEQWRERADSAACCDYSLHVDITRWHESIKEELEALVKEKGVNSFLVFMAYKDRCQCSDSQMYEIFSIIRDLGALAQVHAENGDIVEEEQKRLLELGITGPEGHVLSHPEEVEAEAVYRAVTIAKQANCPLYVTKVMSKGAADAIAQAKRRGVVVFGEPITASLGTDGSHYWSKNWAKAAAFVTSPPVNPDPTTADHLTCLLSSGDLQVTGSAHCTFTTAQKAVGKDNFALIPEGTNGIEERMSMVWEKCVASGKMDENEFVAVTSTNAAKIFNFYPRKGRVAVGSDADLVIWNPKATKIISAKTHNLNVEYNIFEGVECRGAPAVVISQGRVALEDGKMFVTPGAGRFVPRKTFPDFVYKRIKARNRLAEIHGVPRGLYDGPVHEVMVPAKPGSGAPARASCPGKISVPPVRNLHQSGFSLSGSQADDHIARRTAQKIMAPPGGRSNITSLS

SEQ ID NO: 11 - protein, 564 aa, Homo sapiens (Human)
  SITE (1)..(564): Dihydropyrimidinase-related protein 5
MLANSASVRILIKGGKVVNDDCTHEADVYIENGIIQQVGRELMIPGGAKVIDATGKLVIPGGIDTSTHFHQTFMNATCVDDFYHGTKAALVGGTTMIIGHVLPDKETSLVDAYEKCRGLADPKVCCDYALHVGITWWAPKVKAEMETLVREKGVNSFQMFMTYKDLYMLRDSELYQVLHACKDIGAIARVHAENGELVAEGAKEALDLGITGPEGIEISRPEELEAEATHRVITIANRTHCPIYLVNVSSISAGDVIAAAKMQGKVVLAETTTAHATLTGLHYYHQDWSHAAAYVTVPPLRLDTNTSTYLMSLLANDTLNIVASDHRPFTTKQKAMGKEDFTKIPHGVSGVQDRMSVIWERGVVGGKMDENRFVAVTSSNAAKLLNLYPRKGRIIPGADADVVVWDPEATKTISASTQVQGGDFNLYENMRCHGVPLVTISRGRVVYENGVFMCAEGTGKFCPLRSFPDTVYKKLVQREKTLKVRGVDRTPYLGDVAVVVHPGKKEMGTPLADTPTRPVTRHGGMRDLHESSFSLSGSQIDDHVPKRASARILAPPGGRSSGIW

SEQ ID NO: 12 - protein, 355 aa, Homo sapiens (Human)
  SITE (1)..(355): Vasohibin-2
MTGSAADTHRCPHPKGAKGTRSRSSHARPVSLATSGGSEEEDKDGGVLFHVNKSGFPIDSHTWERMWMHVAKVHPKGGEMVGAIRNAAFLAKPSIPQVPNYRLSMTIPDWLQAIQNYMKTLQYNHTGTQFFEIRKMRPLSGLMETAKEMTRESLPIKCLEAVILGIYLTNGQPSIERFPISFKTYFSGNYFHHVVLGIYCNGRYGSLGMSRRAELMDKPLTFRTLSDLIFDFEDSYKKYLHTVKKVKIGLYVPHEPHSFQPIEWKQLVLNVSKMLRADIRKELEKYARDMRMKILKPASAHSPTQVRSRGKSLSPRRRQASPPRRLGRREKSPALPEKKVADLSTLNEVGYQIRI

SEQ ID NO: 13 - protein, 66 aa, Homo sapiens (Human)
  SITE (1)..(66): Small vasohibin-binding protein
MDPPARKEKTKVKESVSRVEKAKQKSAQQELKQRQRAEIYALNRVMTELEQQQFDEFCKQMQPPGE

SEQ ID NO: 14 - protein, 487 aa, Trypanosoma brucei
  SITE (1)..(487): Uncharacterized protein / VASH
MYSSDRVRTVMEQLASMNPYTNAKSLPEVPRPDELSIPRKLSSRLQIPYVQNILNSLSYNFLPNTFFCLEKHRSLQSILFTSKEILAEALPIRCLEATFVGLYLTQDLKDVDRIPLSFKSRAKGRAYHHIVLVIRCNSMYGAVGLSRKSTLMDKPLIYNGLYQLIMEYKKQYEAIGHELVDFKLGLCVNHKRDSRKAPCWRYIAVKLGRSTAPSTSTSPRPQEQQEEVSTNEVDVSLISNSGGCSCGDGIEKMLKCYSDLLCRLSEEYDNLLTSYSRGASNNNQQVCLKDLHDMDNSSAKVENRRRLEELRQGRSPCGSVGTRCKKRSASRSVSKKRAVSQNDVPSTDRSRVVRLSSAVATKKTARREKAAAANYRQPAASDLKQPREFSSASGEKGDGQLPHVSRGTNGNISPERACRAAEEGCAANPGEEVTGERQSVRNPLLSLALPLLDSKGGELGLTTCQATDRWMLPNSAKGANINDVGRS

SEQ ID NO: 15 - protein, 154 aa, Artificial
  Telokin / Homo sapiens
MAMISGLSGRKSSTGSPTSPLNAEKLESEEDVSQAFLEAVAEEKPHVKPYFSKTIRDLEVVEGSAARFDCKIEGYPDPEVVWFKDDQSIRESRHFQIDYDEDGNCSLIISDVCGDDDAKYTCKAVNSLGEATCTAELIVETMEEGEGEGEEEEE

SEQ ID NO: 16 - protein, 159 aa, Artificial
  Telokin/ Mus musculus
MAMISGLSGRKSSTGSPTSPINAEKLESEDDVSQAFLEAVAEEKPHVKPYFSKTIRDLEVVEGSAARFDCKIEGYPDPEVVWFKDDQSIRESRHFQIDYDEDGNCSLIISDVCGDDDAKYTCKAVNSLGEATCTAELIVETMEEGEGEEGGEEEEEEEE

SEQ ID NO: 17 - protein, 154 aa, Artificial
  Fully tyrosinated variant Telokin/Mus musculus
MAMISGLSGRKSSTGSPTSPINAEKLESEDDVSQAFLEAVAEEKPHVKPYFSKTIRDLEVVEGSAARFDCKIEGYPDPEVVWFKDDQSIRESRHFQIDYDEDGNCTLIISDVCGDDDAKYTCKAVNSLGEATCTAELIVETMEEGEGEEEGEEY

SEQ ID NO: 18 - protein, 153 aa, Artificial
  Detyrosinated (D1) variant Telokin/Mus musculus
MAMISGLSGRKSSTGSPTSPINAEKLESEDDVSQAFLEAVAEEKPHVKPYFSKTIRDLEVVEGSAARFDCKIEGYPDPEVVWFKDDQSIRESRHFQIDYDEDGNCTLIISDVCGDDDAKYTCKAVNSLGEATCTAELIVETMEEGEGEEEGEE

SEQ ID NO: 19 - protein, 451 aa, Homo sapiens (Human)
  SITE (1)..(451): Tubulin alpha-1A chain
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 20 - protein, 451 aa, Homo sapiens (Human) [sequence omitted]

SEQ ID NO: 21 - protein, 449 aa, Homo sapiens (Human)
  SITE (1)..(449): Tubulin alpha-1C chain
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLTVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAVAEAWARLDHKFDLMYAKRAFVHWYVGEGMEEGEFSEAREDMAALEKDYEEVGADSADGEDEGEEY

SEQ ID NO: 22 - protein, 450 aa, Homo sapiens (Human)
  SITE (1)..(450): Tubulin alpha-3C/D chain
MRECISIHVGQAGVQIGNACWELYCLEHGIQPDGQMPSDKTIGGGDDSFNTFFSETGAGKHVPRAVFVDLEPTVVDEVRTGTYRQLFHPEQLITGKEDAANNYARGHYTIGKEIVDLVLDRIRKLADLCTGLQGFLIFHSFGGGTGSGFASLLMERLSVDYGKKSKLEFAIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCMLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGVDSVEAEAEEGEEY

SEQ ID NO: 23 - protein, 450 aa, Homo sapiens (Human)
  SITE (1)..(450): Tubulin alpha-3E chain
MRECISIHVGQAGVQIGNACWELYCLEHGIQPDGQMPSDKTIGGGDDSFNTFFSETGAGKHVPRAVFVDLEPTVVDEVRTGTYRQLFHPEQLITGKEDAASNYARGHYTIGKEIVDLVLDRIRKLADLCTGLQGFLIFHSFGGGTGSGFASLLMERLSVDYSKKSKLEFAIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCMLYRGDVVPKDVNAAIATIKTKRTIQFVDWCPTGFKVGINYQPPTVVPGGDLAKVQRAVCMLSNTTAIAEAWARLVHKFDLMYAKWAFVHWYVGEGMEEGEFSEAREDLAALEKDCEEVGVDSVEAEAEEGEAY

SEQ ID NO: 24 - protein, 449 aa, Homo sapiens (Human)
  SITE (1)..(449): Tubulin alpha-8 chain
MRECISVHVGQAGVQIGNACWELFCLEHGIQADGTFDAQASKINDDDSFTTFFSETGNGKHVPRAVMIDLEPTVVDEVRAGTYRQLFHPEQLITGKEDAANNYARGHYTVGKESIDLVLDRIRKLTDACSGLQGFLIFHSFGGGTGSGFTSLLMERLSLDYGKKSKLEFAIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLVTYAPIISAEKAYHEQLSVAEITSSCFEPNSQMVKCDPRHGKYMACCMLYRGDVVPKDVNVAIAAIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGTDSFEEENEGEEF

SEQ ID NO: 25 - protein, 451 aa, Trypanosoma brucei
  SITE (1)..(451): Tubulin alpha chain
MREAICIHIGQAGCQVGNACWELFCLEHGIQPDGAMPSDKTIGVEDDAFNTFFSETGAGKHVPRAVFLDLEPTVVDEVRTGTYRQLFHPEQLISGKEDAANNYARGHYTIGKEIVDLCLDRIRKLADNCTGLQGFLVYHAVGGGTGSGLGALLLERLSVDYGKKSKLGYTVYPSPQVSTAVVEPYNSVLSTHSLLEHTDVAAMLDNEAIYDLTRRNLDIERPTYTNLNRLIGQVVSSLTASLRFDGALNVDLTEFQTNLVPYPRIHFVLTSYAPVISAEKAYHEQLSVSEISNAVFEPASMMTKCDPRHGKYMACCLMYRGDVVPKDVNAAVATIKTKRTIQFVDWSPTGFKCGINYQPPTVVPGGDLAKVQRAVCMIANSTAIAEVFARIDHKFDLMYSKRAFVHWYVGEGMEEGEFSEAREDLAALEKDYEEVGAESADMDGEEDVEEY

SEQ ID NO: 26 - protein, 442 aa, Trypanosoma brucei
  SITE (1)..(442): Tubulin beta chain
MREIVCVQAGQCGNQIGSKFWEVISDEHGVDPTGTYQGDSDLQLERINVYFDEATGGRYVPRSVLIDLEPGTMDSVRAGPYGQIFRPDNFIFGQSGAGNNWAKGHYTEGAELIDSVLDVCCKEAESCDCLQGFQICHSLGGGTGSGMGTLLISKLREQYPDRIMMTFSIIPSPKVSDTVVEPYNTTLSVHQLVENSDESMCIDNEALYDICFRTLKLTTPTFGDLNHLVSAVVSGVTCCLRFPGQLNSDLRKLAVNLVPFPRLHFFMMGFAPLTSRGSQQYRGLSVPELTQQMFDAKNMMQAADPRHGRYLTASALFRGRMSTKEVDEQMLNVQNKNSSYFIEWIPNNIKSSVCDIPPKGLKMAVTFIGNNTCIQEMFRRVGEQFTLMFRRKAFLHWYTGEGMDEMEFTEAESNMNDLVSEYQQYQDATIEEEGEFDEEEQY

SEQ ID NO: 27 - protein, 453 aa, Toxoplasma gondii
  SITE (1)..(453): Tubulin alpha chain
MREVISIHVGQAGIQIGNACWELFCLEHGIQPDGQMPSDKTIGGGDDAFNTFFSETGAGKHVPRCVFLDLEPTVVDEVRTGTYRHLFHPEQLISGKEDAANNFARGHYTIGKEIVDLSLDRIRKLADNCTGLQGFLMFNAVGGGTGSGLGCLLLERLSVDYGKKSKLNFCSWPSPQVSTAVVEPYNSVLSTHSLLEHTDVAVMLDNEAIYDICRRNLDIERPTYTNLNRLIAQVISSLTASLRFDGALNVDVTEFQTNLVPYPRIHFMLSSYAPIISAEKAYHEQLSVAEITNSAFEPASMMAKCDPRHGKYMACCLMYRGDVVPKDVNAAVATIKTKRTIQFVDWCPTGFKCGINYQPPTVVPGGDLAKVMRAVCMISNSTAIAEVFSRMDHKFDLMYAKRAFVHWYVGEGMEEGEFSEAREDLAALEKDYEEVGIETAEGEGEEEGYGDEY

SEQ ID NO: 28 - protein, 450 aa, Spodoptera frugiperda
  SITE (1)..(450): Tubulin alpha chain
MRECISVHVGQAGVQIGNACWELYCLEHGIQPDGQMPTDKTVGGGDDSFNTFFSETGAGKHVPRAVFVDLEPTVVDEVRTGTYRQLFHPEQLITGKEDAANNYARGHYTIGKEIVDLVLDRIRKLADQCTGLQGFLIFHSFGGGTGSGFTSLLMERLSVDYGKKSKLEFAIHPAPQVSTAVVEPYNSILTTHTTLEHPDCAFMVDNEAIYDICRRNLDIERPTYTNLNRLIGQIVSSITASLRFDGALNVDLTEFQTNLVPYPRIHFPLVTYAPVISTEKAYHEQLSVAEITNACFEPANQMVKCDPRHGKYMACCMLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGMDSAEGEGEGAEEY

SEQ ID NO: 29 - protein, 450 aa, Drosophila melanogaster
  SITE (1)..(450): Tubulin alpha chain
MRECISIHVGQAGVQIGNACWELYCLEHGIQPDGQMPSDKTVGGGDDSFNTFFSETGAGKHVPRAVFVDLEPTVVDEVRTGTYRQLFHPEQLITGKEDAANNYARGHYTIGKEIVDLVLDRIRKLADQCTGLQGFLIFHSFGGGTGSGFTSLLMERLSVDYGKKSKLEFAVYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLVTYAPVISAEKAYHEQLSVAEITNACFEPANQMVKVDPRHGKYMACCMLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGMDSGDGEGEGAEEY

SEQ ID NO: 30 - protein, 268 aa, Homo sapiens (Human)
  SITE (1)..(268): Microtubule-associated protein RP/EB family member 1
MAVNVYSTSVTSDNLSRHDMLAWINESLQLNLTKIEQLCSGAAYCQFMDMLFPGSIALKKVKFQAKLEHEYIQNFKILQAGFKRMGVDKIIPVDKLVKGKFQDNFEFVQWFKKFFDANYDGKDYDPVAARQGQETAVAPSLVAPALNKPKKPLTSSSAAPQRPISTQRTAAAPKAGPGVVRKNPGVGNGDDEAAELMQQVNVLKLTVEDLEKERDFYFGKLRNIELICQENEGENDPVLQRIVDILYATDEGFVIPDEGGPQEEQEEY

SEQ ID NO: 31 - protein, 327 aa, Homo sapiens (Human)
  SITE (1)..(327): Microtubule-associated protein RP/EB family member 2
MPGPTQTLSPNGENNNDIIQDNNGTIIPFRKHTVRGERSYSWGMAVNVYSTSITQETMSRHDIIAWVNDIVSLNYTKVEQLCSGAAYCQFMDMLFPGCISLKKVKFQAKLEHEYIHNFKLLQASFKRMNVDKVIPVEKLVKGRFQDNLDFIQWFKKFYDANYDGKEYDPVEARQGQDAIPPPDPGEQIFNLPKKSHHANSPTAGAAKSSPAAKPGSTPSRPSSAKRASSSGSASKSDKDLETQVIQLNEQVHSLKLALEGVEKERDFYFGKLREIELLCQEHGQENDDLVQRLMDILYASEEHEGHTEEPEAEEQAHEQQPPQQEEY

SEQ ID NO: 32 - protein, 281 aa, Homo sapiens (Human)
  SITE (1)..(281): Microtubule-associated protein RP/EB family member 3
MAVNVYSTSVTSENLSRHDMLAWVNDSLHLNYTKIEQLCSGAAYCQFMDMLFPGCVHLRKVKFQAKLEHEYIHNFKVLQAAFKKMGVDKIIPVEKLVKGKFQDNFEFIQWFKKFFDANYDGKDYNPLLARQGQDVAPPPNPGDQIFNKSKKLIGTAVPQRTSPTGPKNMQTSGRLSNVAPPCILRKNPPSARNGGHETDAQILELNQQLVDLKLTVDGLEKERDFYFSKLRDIELICQEHESENSPVISGIIGILYATEEGFAPPEDDEIEEHQQEDQDEY

SEQ ID NO: 33 - protein, 51 aa, Artificial
  50 amino acids Last C-ter alpha-tubulin 1A/1B
KRAFVHWYVGEGMEEGEFSEAREDMAALEKDYEEVGVDSVEGEGEEEGEEY

SEQ ID NO: 34 - protein, 12 aa, Artificial
  12 amino acids Last C-ter alpha-tubulin 1A/1B
VEGEGEEEGEEY

SEQ ID NO: 35 - protein, 12 aa, Artificial
  12 amino acids Last C-ter alpha-tubulin 3C
SVEAEAEEGEEY

SEQ ID NO: 36 - protein, 12 aa, Artificial
  12 amino acids Last C-ter alpha-tubulin 1C
DSADGEDEGEEY

SEQ ID NO: 37 - protein, 12 aa, Artificial
  12 amino acids Last C-ter alpha-tubulin 3E
SVEAEAEEGEAY

SEQ ID NO: 38 - protein, 8 aa, Artificial
  8 amino acids last C-ter alpha-tubulin 1A/1B
GEEEGEEY

SEQ ID NO: 39 - protein, 11 aa, Artificial
  11 amino acids  last C-ter alpha-tubulin 1A/1B
EGEGEEEGEEY

SEQ ID NO: 40 - protein, 8 aa, Artificial
  7 amino acids last C-ter alpha tubulin from Spodoptera frugiperda without the most C-terminal Y (DeTyr)
GEGEGAEE